(19) United States
(12) Patent Application Publication
(10) Pub. No.: US 2015/0065447 A1
(43) Pub. Date: Mar. 5, 2015

(54) SURVIVAL BENEFIT IN PATIENTS WITH SOLID TUMORS WITH ELEVATED C-REACTIVE PROTEIN LEVELS

(71) Applicant: Incyte Corporation, Wilmington, DE (US)

(72) Inventor: Victor Sandor, Kennett Square, PA (US)

(73) Assignee: Incyte Corporation, Wilmington, DE (US)

(21) Appl. No.: 14/463,232

(22) Filed: Aug. 19, 2014

Related U.S. Application Data
(60) Provisional application No. 61/867,982, filed on Aug. 20, 2013.

(51) Int. Cl. C07D 495/14; A61K 45/06; A61K 31/7068; G01N 33/574; C07D 403/14; A61K 31/4155; C07D 487/04; A61K 31/519; A61K 31/437

(52) U.S. Cl. CPC C07D 495/14; A61K 31/519; A61K 45/06; A61K 31/7068; A61K 31/437; C07D 403/14; A61K 31/4155; C07D 487/04; G01N 33/57496; G01N 2800/52; G01N 2333/4737
USPC 514/49; 514/265.1; 514/293; 514/210.2; 435/7.92; 436/501

(57) ABSTRACT

This application relates to methods of increasing survival or progression-free survival in a patient with a solid tumor, wherein the patient has an elevated serum concentration of C-reactive protein (CRP), by administering a JAK inhibitor or an inhibitor of IL-6 signaling to the patient, as well as methods of predicting survival benefit in these patients from such therapy.

This application claims the benefit of priority of U.S. Prov. Appl. No. 61/867,982, filed Aug. 20, 2013, which is incorporated herein by reference in its entirety.

### TECHNICAL FIELD

This application relates to methods of increasing survival or progression-free survival in a patient with a solid tumor, wherein the patient has an elevated serum concentration of C-reactive protein (CRP), by administering a JAK inhibitor or an inhibitor of IL-6 signaling to the patient, as well as methods of predicting survival benefit in these patients from such therapy.

### BACKGROUND

Janus Kinases (JAKs) play an important role in signal transduction following cytokine and growth factor binding to their receptors. Aberrant activation of JAKs, through either aberrant or excessive cytokine signaling or through intracellular mechanisms causing pathway dysregulation has been associated with increased malignant cell proliferation and survival.

There are multiple mechanisms through which inflammatory cytokines can impact tumor growth and survival. Cytokines are key molecules controlling autocrine or paracrine communications within and between tumor cells and tumor cells and their surrounding stromal environment. While under some circumstances, endogenous cytokines may orchestrate host responses against the tumor, the cytokine network also contributes to tumor growth, progression and host immuno-suppression. In addition, inflammatory cytokines have been implicated as key mediators of the catabolic state and cachexia associated with cancer, and they can, therefore, impact the course of patients with cancer through this mechanism as well as direct effects on tumor cells. C-reactive protein (CRP) is a protein that can be measured in serum and is a broad measure of systemic inflammatory response and is associated with elevated levels of cytokines, in particular, IL-6. Elevated CRP has been associated with a poor prognosis and poor responsiveness to conventional therapies in a broad range of tumors (McMillan, D. C., Cancer Treatment Reviews 39 (2013) 534-540). There is a medical need to improve the treatment of patients with cancer with this poor prognostic factor. This invention is directed to this need and others.

### SUMMARY

The present application provides, inter alia, a method of increasing survival or progression-free survival in a patient that has a solid tumor, wherein the patient has an elevated serum concentration of C-reactive protein (CRP), comprising administering a Janus Kinase (JAK) inhibitor or an inhibitor of IL-6 signaling to the patient, wherein the administering increases survival or progression-free survival of the patient.

The present application also provides a method of increasing survival or progression-free survival in a patient that has a solid tumor, wherein the patient has a modified Glasgow Prognostic Score (mGPS) of 1 or 2, comprising administering a JAK inhibitor or an inhibitor of IL-6 signaling to the patient, wherein the administering increases survival or progression-free survival of the patient.

The present application further provides a method of treating a solid tumor in a patient in need thereof, wherein the patient modified Glasgow Prognostic Score (mGPS) of 1 or 2, comprising administering a Janus Kinase (JAK) inhibitor or an inhibitor of IL-6 signaling to the patient. The present application further provides a method of treating a solid tumor, comprising:

(a) selecting a patient having the solid tumor with a serum concentration of C-reactive protein (CRP) that is equal to or greater than a median baseline serum concentration of CRP for a population of patients with the solid tumor;

(b) administering to the patient a therapeutically effective amount of a JAK inhibitor or an inhibitor of IL-6 signaling.

The present application also provides a method of treating a solid tumor, comprising:

(a) selecting a patient having the solid tumor with a serum concentration of C-reactive protein (CRP) that is equal to or greater than about 10 μg/mL;

(b) administering to the patient a therapeutically effective amount of a JAK inhibitor or an inhibitor of IL-6 signaling.

The present application further provides a method of treating a solid tumor, comprising:

(a) selecting a patient having the solid tumor with a modified Glasgow Prognostic Score of 1 or 2;

(b) administering to the patient a therapeutically effective amount of a JAK inhibitor or an inhibitor of IL-6 signaling.

The present application also provides a method of predicting a benefit to a patient having a solid tumor of treatment using a JAK inhibitor or an inhibitor of IL-6 signaling, comprising comparing said serum concentration of C-reactive protein (CRP) of the patient to a baseline serum concentration of CRP of a population of patients having the solid tumor, wherein the serum CRP concentration in the patient of equal to or greater than the baseline serum concentration is indicative of a benefit to the patient of the treatment using the JAK inhibitor or an inhibitor of IL-6 signaling.

The present application further provides a JAK inhibitor or an inhibitor of IL-6 signaling for use as described in any of the methods described by the embodiments herein.

The present application provides the use of a JAK inhibitor or an inhibitor of IL-6 signaling for the preparation of a medicament for use in any of the methods described by the embodiments herein.

### DESCRIPTION OF DRAWINGS

FIG. 1 depicts the Kaplan-Meier analysis of overall survival for patients whose baseline CRP was less than or equal to 13 μg/mL (survival distribution function versus days, for Arm 1 and Arm 2).

FIG. 2 depicts the Kaplan-Meier analysis of overall survival for patients whose baseline CRP was greater than 13 μg/mL (survival distribution function versus days, for Arm 1 and Arm 2).

FIG. 3 depicts the Kaplan-Meier analysis of progression-free survival for patients whose baseline CRP was less than or equal to 13 μg/mL (survival distribution function versus days to progression, for Arm 1 and Arm 2).

FIG. 4 depicts the Kaplan-Meier analysis of progression-free survival for patients whose baseline CRP was greater than 13 μg/mL (survival distribution function versus days to progression, for Arm 1 and Arm 2).

FIG. 5(A)-(C) depict Kaplan-Meier curves for overall survival by baseline mGPS (A, mGPS=0; B, mGPS=1; and C, mGPS=2) (y-axis is survival distribution function; and y-axis is survival days).

### DETAILED DESCRIPTION

The present application provides a method of increasing survival or progression-free survival in a patient that has a solid tumor, wherein the patient has an elevated serum concentration of C-reactive protein (CRP), comprising administering a JAK inhibitor or an inhibitor of IL-6 signaling to the patient, wherein the administering increases survival or progression-free survival of the patient.

In some embodiments, the method further comprises selecting a patient with an elevated serum concentration of C-reactive protein prior to the administering.

In some embodiments, an elevated serum concentration of CRP is a serum concentration that is equal to or greater than a median baseline serum concentration of CRP for a population of patients with the solid tumor (i.e., as measured by a CRP assay).

In some embodiments, an elevated serum concentration of CRP is one that is equal to or greater than about 10 μg/mL.

In some embodiments, an elevated serum concentration of CRP is one that is equal to or greater than 2 times the upper limit of the normal value.

In some embodiments, an elevated serum concentration of CRP is one that is equal to or greater than 2.5 times the upper limit of the normal value.

In some embodiments, an elevated serum concentration of CRP is one that is equal to or greater than 3 times the upper limit of the normal value.

In some embodiments, an elevated serum concentration of CRP is one that is equal to or greater than 3.5 times the upper limit of the normal value.

In some embodiments, an elevated serum concentration of CRP is one that is equal to or greater than 4 times the upper limit of the normal value.

The present application further provides a method of treating a solid tumor, comprising:

(a) selecting a patient having the solid tumor with a serum concentration of C-reactive protein (CRP) that is equal to or greater than a median baseline serum concentration of CRP for a population of patients with the solid tumor;

(b) administering to the patient a therapeutically effective amount of a JAK inhibitor or an inhibitor of IL-6 signaling.

The present application also provides a method of treating a solid tumor, comprising:

(a) selecting a patient having the solid tumor with a serum concentration of C-reactive protein (CRP) that is equal to or greater than about 10 μg/mL;

(b) administering to the patient a therapeutically effective amount of a JAK inhibitor or an inhibitor of IL-6 signaling.

In some embodiments, the administering increases survival of the patient.

In some embodiments, the administering increases progression-free survival of the patient.

In some embodiments, the serum concentration of CRP is equal to or greater than about 13 μg/mL.

In some embodiments, the serum concentration of CRP is equal to or greater than about 10 μg/mL.

In some embodiments, the present application provides a method of treating a solid tumor, comprising:

(a) selecting a patient having the solid tumor with a modified Glasgow Prognostic Score of 1 or 2;

(b) administering to the patient a therapeutically effective amount of a JAK inhibitor or an inhibitor of IL-6 signaling.

In some embodiments, the administering increases survival of the patient.

In some embodiments, the administering increases progression-free survival of the patient.

The modified Glasgow Prognosis Score (mGPS) is described in McMillian, Cancer Treatment Reviews, 39 (5):534-540 (2013), which is incorporated herein by reference in its entirety (and in particular, the scores as shown in Table 1, which is reproduced below).

| Modified Glasgow Prognostic Score | Score |
|---|---|
| C-reactive protein ≦ 10 mg/L | 0 |
| C-reactive protein > 10 mg/l and albumin ≧ 35 g/L | 1 |
| C-reactive protein > 10 mg/l and albumin < 35 g/L | 2 |

The serum CRP concentrations can be measured using a standard commercial assay or, alternatively, a Rules Based Medicines (RBM) assay. A commercial clinical assay for CRP includes without limitation the Quest Diagnostics C-Reactive Protein (CRP) test or Labcorp c-Reactive Protein High Sensitivity test. The RBM assay includes without limitation the RBM multiplexed Luminex®) commercial assay (Myriad RBM). The commercial clinical assays can be correlated. For example, it is believed that a 10 μg/mL serum concentration in an RBM assay correlates to about a 10 μg/mL in a clinical assay.

CRP tests are approved by FDA under a 510K process and most of the available tests utilized a 510K substantial equivalence test based on a predicate test with established standards for analytical validation of the individual test as well as the analytic platform on which the test is conducted. Conventional CRP assays carry a general indication for use for evaluation of infection, tissue injury, and inflammatory disorders. These assays provide information for the diagnosis, therapy, and monitoring of inflammatory diseases (FDA Guidance for Industry—Review Criteria for Assessment of C Reactive Protein (CRP), High Sensitivity C-Reactive Protein (h5CRP) and Cardiac C-Reactive Protein (cCRP) Assays, www.fda.gov/medicaldevices/deviceregulationandguidance/guidancedocuments/ucm077167.htm Accessed Sep. 17, 201). CRP is one of the cytokine-induced “acute-phase” proteins whose blood levels rise during a general, unspecific response to infections and non-infectious inflammatory processes (Pepys and Hirschfield, J Clin Invest 2003 111:1805-1812). CRP reflects ongoing inflammation and/or tissue damage much more accurately than do other laboratory parameters of the acute-phase response, such as plasma viscosity and the erythrocyte sedimentation rate. Importantly, acute-phase CRP values show no diurnal variation and are unaffected by eating. Liver failure impairs CRP production, but no other intercurrent pathologies and very few drugs reduce CRP values unless they also affect the underlying pathology providing the acute-phase stimulus. The CRP concentration is thus a very useful nonspecific biochemical marker of inflammation (Pepys and Hirschfield 2003). For conventional CRP assays, test values are typically considered to be clinically significant at levels above 10 mg/L (FDA CRP Guidance).

The use of CRP as part of the mGPS to assess inflammation related to cancer is well established in the medical literature (McMillan, Cancer Treat Rev 2013; 39:534-540), and falls within the approved labeling and intended use of conventional CRP assays. The cutoff value distinguishing mGPS 0 from mGPS 1 and 2 is the same as the value generally accepted as clinically significant. CRP used as part of the mGPS to determine study eligibility will be conducted at a central laboratory using a single FDA approved assay system for all study subjects.

As used herein the term “JAK inhibitor” is intended to mean compounds inhibit at least JAK1 and/or JAK2. In some embodiments, the JAK inhibitor is JAK2 inhibitor. In some embodiments, the JAK inhibitor is a JAK1 inhibitor.

In some embodiments, the JAK inhibitor can also inhibit other members of the Janus kinase family (i.e., JAK3 or TYK2). In some embodiments, the JAK inhibitor is selective. By “selective” is meant that the compound binds to or inhibits a JAK1 and/or JAK2 with greater affinity or potency, respectively, compared to at least one other JAK (e.g., JAK2, JAK3 and/or TYK2). In some embodiments, the JAK inhibitor is selective for JAK1 and JAK2 over JAK3 and TYK2. In some embodiments, the compounds of the invention are selective inhibitors of JAK 1 over JAK2, JAK3, and TYK2. Selectivity can be at least about 5-fold, at least about 10-fold, at least about 20-fold, at least about 50-fold, at least about 100-fold, at least about 200-fold, at least about 500-fold or at least about 1000-fold. Selectivity can be measured by methods routine in the art. In some embodiments, selectivity can be tested at the Km of each enzyme. In some embodiments, selectivity of compounds for JAK1 and/or JAK2 can be determined by the cellular ATP concentration.

In some embodiments, the methods comprise administering a JAK1 and/or JAK2 inhibitor to the patient.

In some embodiments, the methods comprise administering a JAK1 inhibitor to the patient.

In some embodiments, the methods comprise administering a JAK2 inhibitor to the patient.

In some embodiments, the methods comprise administering an inhibitor of IL-6 signaling to the patient.

In some embodiments, the JAK inhibitor is ruxolitinib, or a pharmaceutically acceptable salt thereof.

In some embodiments, the JAK inhibitor is ruxolitinib phosphate.

In some embodiments, the JAK inhibitor is a selective JAK1 inhibitor. As used herein, a “selective JAK1 inhibitor” is an inhibitor of JAK1 which is selective for JAK1 over JAK2, JAK3 and TYK2. In some embodiments, the compounds or salts are about 10-fold more selective for JAK1 over JAK2. In some embodiments, the compounds or salts are about 10-fold, about 15-fold, or about 20-fold more selective for JAK1 over JAK2 as calculated by measuring IC₅₀ at 1 mM ATP (e.g., see Example A).

In some embodiments, the selective JAK1 inhibitor is a compound of Table A, or a pharmaceutically acceptable salt thereof. The compounds in Table A are selective JAK1 inhibitors (selective over JAK2, JAK3, and TYK2). The IC₅₀s obtained by the method of Assay A at 1 mM ATP are shown in Table A.

TABLE A

| | | | | JAK1 | |
|---|---|---|---|---|---|
| | | | | IC₅₀ | JAK2/ |
| # | Prep. | Name | Structure | (nM) | JAK1 |
| 1 | Example J1 herein | ((2R,5S)-5-{2-[(1R)- 1-hydroxyethyl]-1H- imidazo[4,5- d]thieno[3,2- b]pyridin-1- yl}tetrahydro-2H- pyran-2- yl)acetonitrile | 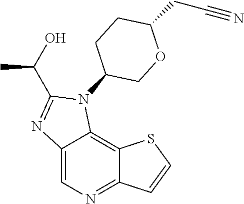 [SMILES: C[C@@H](O)c1nc2cnc3ccsc3c2n1[C@H]1CC[C@H](CC#N)OC1] | ++ | >10 |
| 2 | Example J2 herein | 4-[3-(cyanomethyl)- 3-(3′,5′-dimethyl- 1H,1′H-4,4′- bipyrazol-1- yl)azetidin-1-yl]-2,5- difluoro-N-[(1S)- 2,2,2-trifluoro-1- methylethyl] benzamide | 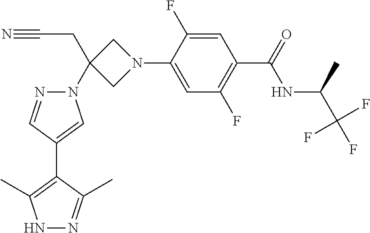 [SMILES: Cc1n[nH]c(C)c1-c1cnn(C2(CC#N)CN(c3cc(F)c(C(=O)N[C@@H](C)C(F)(F)F)cc3F)C2)c1] | +++ | >10 |
| 3 | US 2010/ 0298334 (Example 2)^{a} | 3-[1-(6- chloropyridin-2- yl)pyrrolidin-3-yl]-3- [4-(7H-pyrrolo[2,3- d]pyrimidin-4-yl)- 1H-pyrazol-1- yl]propanenitrile | 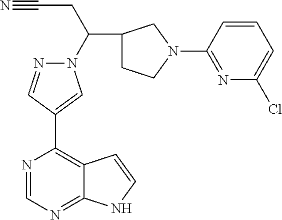 [SMILES: N#CCC(C1CCN(c2cccc(Cl)n2)C1)n1cc(-c2ncnc3[nH]ccc23)cn1] | + | >10 |
| 4 | US 2010/ 0298334 (Example 13c) | 3-(1- [1,3]oxazolo[5,4- b]pyridin-2- ylpyrrolidin-3-yl)-3- [4-(7H-pyrrolo[2,3- d]pyrimidin-4-yl)- 1H-pyrazol-1- yl]propanenitrile | 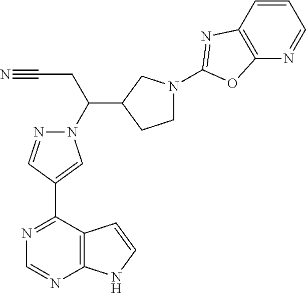 [SMILES: N#CCC(C1CCN(c2nc3cccnc3o2)C1)n1cc(-c2ncnc3[nH]ccc23)cn1] | + | >10 |
| 5 | US 2011/ 0059951 (Example 12) | 4-[(4-{3-cyano-2-[4- (7H-pyrrolo[2,3- d]pyrimidin-4-yl)- 1H-pyrazol-1- yl]propyl}piperazin- 1-yl)carbonyl]-3- fluorobenzonitrile | 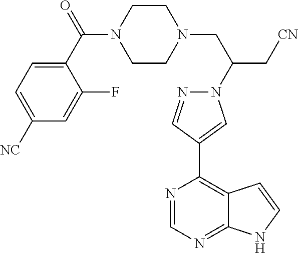 [SMILES: N#CCC(CN1CCN(C(=O)c2ccc(C#N)cc2F)CC1)n1cc(-c2ncnc3[nH]ccc23)cn1] | + | >10 |
| 6 | US 2011/ 0059951 (Example 13) | 4-[(4-{3-cyano-2-[3- (7H-pyrrolo[2,3- d]pyrimidin-4-yl)- 1H-pyrrol-1- yl]propyl}piperazin- l-yl)carbonyl]-3- fluorobenzonitrile | 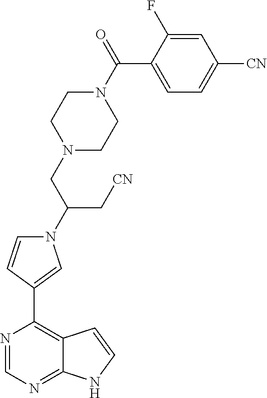 [SMILES: N#CCC(CN1CCN(C(=O)c2ccc(C#N)cc2F)CC1)n1ccc(-c2ncnc3[nH]ccc23)c1] | + | >10 |
| 7 | US 2011/ 0224190 (Example 1) | {l-{l-[3-Fluoro-2- (trifluoromethyl) isonicotinoyl] piperidin-4- yl}-3-[4-(7H- pyrrolo[2,3- d]pyrimidin-4-yl)- 1H-pyrazol-1- yl]azetidin-3- yl}acetonitrile | 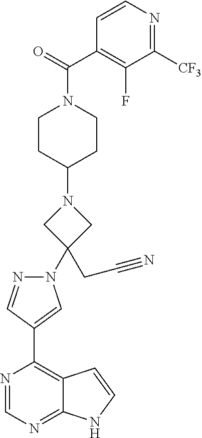 [SMILES: N#CCC1(n2cc(-c3ncnc4[nH]ccc34)cn2)CN(C2CCN(C(=O)c3ccnc(C(F)(F)F)c3F)CC2)C1] | + | >10 |
| 8 | US 2011/ 0224190 (Example 154) | 4-{3-(Cyanomethyl)- 3-[4-(7H-pyrrolo[2,3- d]pyrimidin-4-yl)- 1H-pyrazol-1- yl]azetidin-1-yl}-N- [4-fluoro-2- (trifluoromethyl) phenyl]piperidine-1- carboxamide | 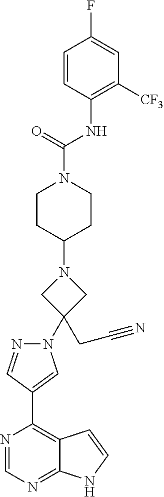 [SMILES: N#CCC1(n2cc(-c3ncnc4[nH]ccc34)cn2)CN(C2CCN(C(=O)Nc3ccc(F)cc3C(F)(F)F)CC2)C1] | + | >10 |
| 9 | US 2011/ 0224190 (Example 85) | [3-[4-(7H- pyrrolo[2,3- d]pyrimidin-4-yl)- 1H-pyrazol-1-yl]-1- (l-{[2- (trifluoromethyl) pyrimidin-4- yl]carbonyl}piperidin- 4-yl)azetidin-3- yl]acetonitrile | 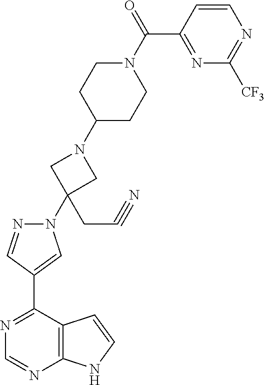 [SMILES: N#CCC1(n2cc(-c3ncnc4[nH]ccc34)cn2)CN(C2CCN(C(=O)c3ccnc(C(F)(F)F)n3)CC2)C1] | + | >10 |
| 10 | US 2012/ 0149681 (Example 7b) | [trans-1-[4-(7H- pyrrolo[2,3- d]pyrimidin-4-yl)- 1H-pyrazol-1-yl]-3- (4-{[2- (trifluoromethyl) pyrimidin-4- yl]carbonyl} piperazin-1- yl)cyclobutyl] acetonitrile | 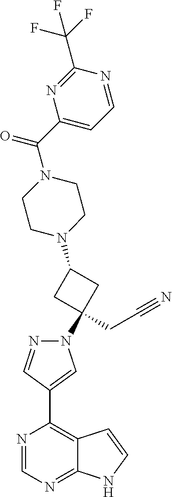 [SMILES: N#CC[C@]1(n2cc(-c3ncnc4[nH]ccc34)cn2)C[C@H](N2CCN(C(=O)c3ccnc(C(F)(F)F)n3)CC2)C1] | + | >10 |
| 11 | US 2012/ 0149681 (Example 157) | {trans-3-(4-{[4-[(3- hydroxyazetidin-1- yl)methyl]-6- (trifluoromethyl) pyridin-2- yl]oxy}piperidin-1- yl)-1-[4-(7H- pyrrolo[2,3- d]pyrimidin-4-yl)- 1H-pyrazol-1- yl]cyclobutyl} acetonitrile | 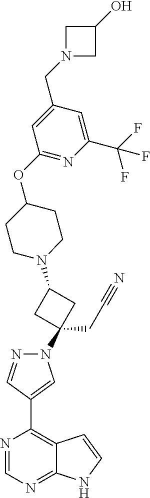 [SMILES: N#CC[C@]1(n2cc(-c3ncnc4[nH]ccc34)cn2)C[C@H](N2CCC(Oc3cc(CN4CC(O)C4)cc(C(F)(F)F)n3)CC2)C1] | + | >10 |
| 12 | US 2012/ 0149681 (Example 161) | {trans-3-(4-{[4- {[(2S)-2- (hydroxymethyl) pyrrolidin-1-yl]methyl}- 6- (trifluoromethyl) pyridin-2- yl]oxy}piperidin-1- yl)-1-[4-(7H- pyrrolo[2,3- d]pyrimidin-4-yl)- 1H-pyrazol-1- yl]cyclobutyl} acetonitrile | 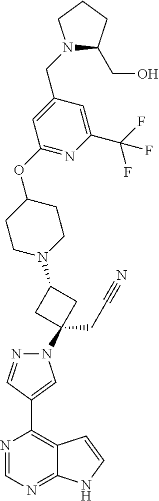 [SMILES: N#CC[C@]1(n2cc(-c3ncnc4[nH]ccc34)cn2)C[C@H](N2CCC(Oc3cc(CN4CCC[C@H]4CO)cc(C(F)(F)F)n3)CC2)C1] | + | >10 |
| 13 | US 2012/ 0149681 (Example 162) | {trans-3-(4-{[4- {[(2R)-2- (hydroxymethyl) pyrrolidin-1-yl] methyl}-6- (trifluoromethyl) pyridin-2- yl]oxy}piperidin-1- yl)-1-[4-(7H- pyrrolo[2,3- d]pyrimidin-4-yl)- 1H-pyrazol-1- yl]cyclobutyl} acetonitrile | 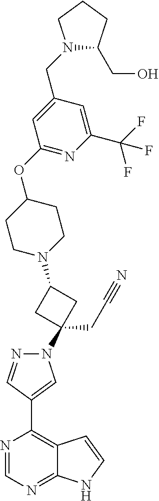 [SMILES: N#CC[C@]1(n2cc(-c3ncnc4[nH]ccc34)cn2)C[C@H](N2CCC(Oc3cc(CN4CCC[C@@H]4CO)cc(C(F)(F)F)n3)CC2)C1] | + | >10 |
| 14 | US 2012/ 0149682 (Example 20)^{b} | 4-(4-{3- [(dimethylamino) methyl]-5- fluorophenoxy} piperidin-1-yl)-3-[4- (7H-pyrrolo[2,3- d]pyrimidin-4-yl)- 1H-pyrazol-1- yl]butanenitrile | 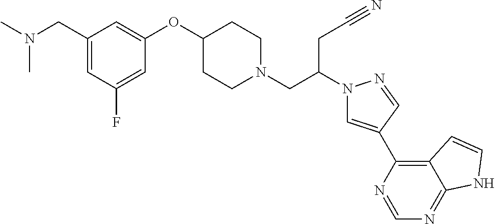 [SMILES: CN(C)Cc1cc(F)cc(OC2CCN(CC(CC#N)n3cc(-c4ncnc5[nH]ccc45)cn3)CC2)c1] | + | >10 |
| 15 | US 2013/ 0018034 (Example 18) | 5-{3-(cyanomethyl)- 3-[4-(7H-pyrrolo[2,3- d]pyrimidin-4-yl)- 1H-pyrazol-1- yl]azetidin-1-yl}-N- isopropylpyrazine-2- carboxamide | 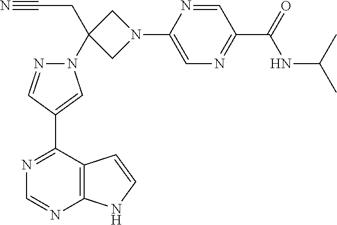 [SMILES: CC(C)NC(=O)c1cnc(N2CC(CC#N)(n3cc(-c4ncnc5[nH]ccc45)cn3)C2)cn1] | + | >10 |
| 16 | US 2013/ 0018034 (Example 28) | 4-{3-(cyanomethyl)- 3-[4-(7H-pyrrolo[2,3- d]pyrimidin-4-yl)- 1H-pyrazol-1- yl]azetidin-1-yl}-2,5- difluoro-N-[(1S)- 2,2,2-trifluoro-1- methylethyl] benzamide | 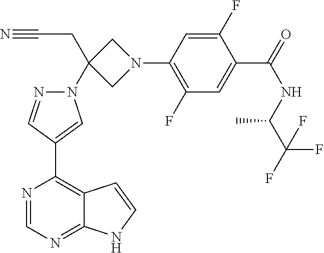 [SMILES: C[C@H](NC(=O)c1cc(F)c(N2CC(CC#N)(n3cc(-c4ncnc5[nH]ccc45)cn3)C2)cc1F)C(F)(F)F] | + | >10 |
| 17 | US 2013/ 0018034 (Example 34) | 5-{3-(cyanomethyl)- 3-[4-(1H-pyrrolo[2,3- b]pyridin-4-yl)-1H- pyrazol-1-yl]azetidin- 1-yl}-N- isopropylpyrazine-2- carboxamide | 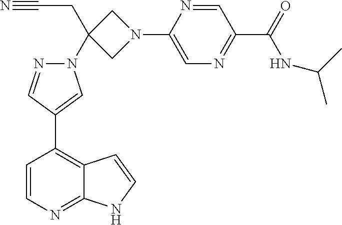 [SMILES: CC(C)NC(=O)c1cnc(N2CC(CC#N)(n3cc(-c4ccnc5[nH]ccc45)cn3)C2)cn1] | + | >10 |
| 18 | US 2013/ 0045963 (Example 45) | (l-(cis-4-{[6-(2- hydroxyethyl)-2- (trifluoromethyl) pyrimidin-4- yl]oxy}cyclohexyl)- 3-[4-(7H-pyrrolo[2,3- d]pyrimidin-4-yl)- 1H-pyrazol-1- yl]azetidin-3- yl}acetonitrile | 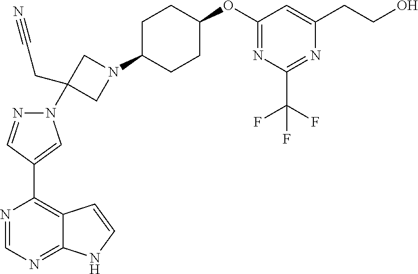 [SMILES: N#CCC1(n2cc(-c3ncnc4[nH]ccc34)cn2)CN([C@H]2CC[C@@H](Oc3cc(CCO)nc(C(F)(F)F)n3)CC2)C1] | + | >10 |
| 19 | US 2013/ 0045963 (Example 65) | {l-(cis-4-{[4- [(ethylamino)methyl]- 6- (trifluoromethyl) pyridin-2- yl]oxy}cyclohexyl)- 3-[4-(7H-pyrrolo[2,3- d]pyrimidin-4-yl)- 1H-pyrazol-1- yl]azetidin-3- yl}acetonitrile | 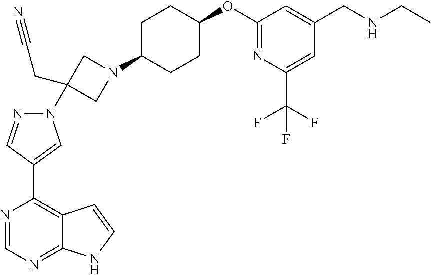 [SMILES: CCNCc1cc(O[C@H]2CC[C@@H](N3CC(CC#N)(n4cc(-c5ncnc6[nH]ccc56)cn4)C3)CC2)nc(C(F)(F)F)c1] | + | >10 |
| 20 | US 2013/ 0045963 (Example 69) | {1-(cis-4-{[4-(1- hydroxy-1- methylethyl)-6- (trifluoromethyl) pyridin-2- yl]oxy}cyclohexyl)- 3-[4-(7H-pyrrolo[2,3- d]pyrimidin-4-yl)- 1H-pyrazol-1- yl]azetidin-3- yl}acetonitrile | 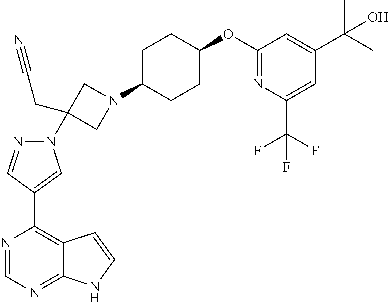 [SMILES: CC(C)(O)c1cc(O[C@H]2CC[C@@H](N3CC(CC#N)(n4cc(-c5ncnc6[nH]ccc56)cn4)C3)CC2)nc(C(F)(F)F)c1] | + | >10 |
| 21 | US 2013/ 0045963 (Example 95) | {1-(cis-4-{[4-{[(3R)- 3-hydroxypyrrolidin- 1-yl]methyl}-6- (trifluoromethyl) pyridin-2- yl]oxy}cyclohexyl)- 3-[4-(7H-pyrrolo[2,3- d]pyrimidin-4-yl)- 1H-pyrazol-1- yl]azetidin-3- yl}acetonitrile | 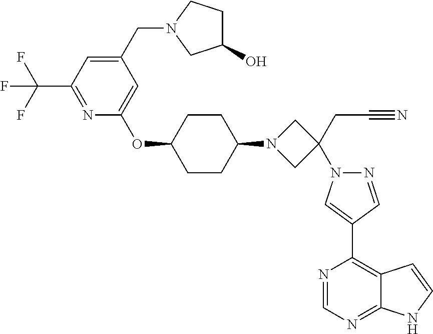 [SMILES: N#CCC1(n2cc(-c3ncnc4[nH]ccc34)cn2)CN([C@H]2CC[C@@H](Oc3cc(CN4CC[C@@H](O)C4)cc(C(F)(F)F)n3)CC2)C1] | + | >10 |
| 22 | US 2013/ 0045963 (Example 95) | {1-(cis-4-{[4-{[(3S)- 3-hydroxypyrrolidin- 1-yl]methyl}-6- (trifluoromethyl) pyridin-2- yl]oxy}cyclohexyl)- 3-[4-(7H-pyrrolo[2,3- d]pyrimidin-4-yl)- 1H-pyrazol-1- yl]azetidin-3- yl}acetonitrile | 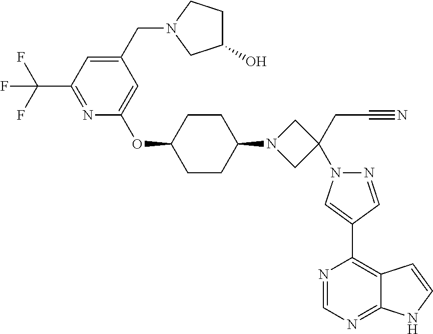 [SMILES: N#CCC1(n2cc(-c3ncnc4[nH]ccc34)cn2)CN([C@H]2CC[C@@H](Oc3cc(CN4CC[C@H](O)C4)cc(C(F)(F)F)n3)CC2)C1] | + | >10 |
| 23 | US 2014/ 0005166 (Example 1) | {trans-3-(4-{[4- ({[(1S)-2-hydroxy-1- methylethyl]amino} methyl)-6- (trifluoromethyl) pyridin-2- yl]oxy}piperidin-1- yl)-1-[4-(7H- pyrrolo[2,3- d]pyrimidin-4-yl)- 1H-pyrazol-1- yl]cyclobutyl} acetonitrile | 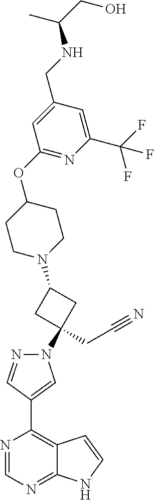 [SMILES: C[C@@H](CO)NCc1cc(OC2CCN([C@H]3C[C@](CC#N)(n4cc(-c5ncnc6[nH]ccc56)cn4)C3)CC2)nc(C(F)(F)F)c1] | + | >10 |
| 24 | US 2014/ 0005166 (Example 14) | {trans-3-(4-{[4- ({[(2R)-2- hydroxypropyl]amino} methyl)-6- (trifluoromethyl) pyridin-2- yl]oxy}piperidin-1- yl)-1-[4-(7H- pyrrolo[2,3- d]pyrimidin-4-yl)- 1H-pyrazol-1- yl]cyclobutyl} acetonitrile | 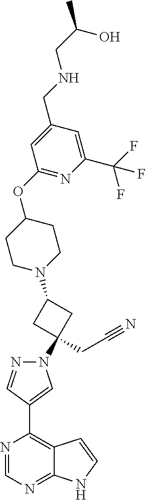 [SMILES: C[C@@H](O)CNCc1cc(OC2CCN([C@H]3C[C@](CC#N)(n4cc(-c5ncnc6[nH]ccc56)cn4)C3)CC2)nc(C(F)(F)F)c1] | + | >10 |
| 25 | US 2014/ 0005166 (Example 15) | {trans-3-(4-{[4- ({[(2S)-2- hydroxypropyl]amino} methyl)-6- (trifluoromethyl) pyridin-2- yl]oxy}piperidin-1- yl)-1-[4-(7H- pyrrolo[2,3- d]pyrimidin-4-yl)- 1H-pyrazol-1- yl]cyclobutyl} acetonitrile | 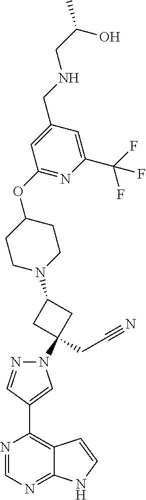 [SMILES: C[C@H](O)CNCc1cc(OC2CCN([C@H]3C[C@](CC#N)(n4cc(-c5ncnc6[nH]ccc56)cn4)C3)CC2)nc(C(F)(F)F)c1] | + | >10 |
| 26 | US 2014/ 0005166 (Example 20) | {trans-3-(4-{[4-(2- hydroxyethyl)-6- (trifluoromethyl) pyridin-2- yl]oxy}piperidin-1- yl)-1-[4-(7H- pyrrolo[2,3- d]pyrimidin-4-yl)- 1H-pyrazol-1- yl]cyclobutyl} acetonitrile | 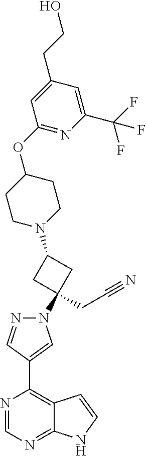 [SMILES: N#CC[C@]1(n2cc(-c3ncnc4[nH]ccc34)cn2)C[C@H](N2CCC(Oc3cc(CCO)cc(C(F)(F)F)n3)CC2)C1] | + | >10 |

+ means <10 nM (see Example A for assay conditions)
++ means ≦ 100 nM (see Example A for assay conditions)
+++ means ≦ 300 nM (see Example A for assay conditions)
^{a}Data for enantiomer 1
^{b}Data for enantiomer 2

In some embodiments, the selective JAK1 inhibitor is {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof.

In some embodiments, the selective JAK1 inhibitor is {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile adipic acid salt.

In some embodiments, the selective JAK1 inhibitor is 4-{3-(cyanomethyl)-3-[4-(7H-pyrrolo[2,3-d]pyrimidin-4-yl)-1H-pyrazol-1-yl]azetidin-1-yl}-2,5-difluoro-N-[(1S)-2,2,2-trifluoro-1-methylethyl]benzamide, or a pharmaceutically acceptable salt thereof.

In some embodiments, the selective JAK1 inhibitor is selected from (R)-3-[1-(6-chloropyridin-2-yl)pyrrolidin-3-yl]-3-[4-(7H-pyrrolo[2,3-d]pyrimidin-4-yl)-1H-pyrazol-1-yl]propanenitrile, (R)-3-(1-[1,3]oxazolo[5,4-b]pyridin-2-ylpyrrolidin-3-yl)-3-[4-(7H-pyrrolo[2,3-d]pyrimidin-4-yl)-1H-pyrazol-1-yl]propanenitrile, (R)-4-[(4-{3-cyano-2-[4-(7H-pyrrolo[2,3-d]pyrimidin-4-yl)-1H-pyrazol-1-yl]propyl}piperazin-1-yl)carbonyl]-3-fluorobenzonitrile, (R)-4-[(4-{3-cyano-2-[3-(7H-pyrrolo[2,3-d]pyrimidin-4-yl)-1H-pyrrol-1-yl]propyl}piperazin-1-yl)carbonyl]-3-fluorobenzonitrile, or (R)-4-(4-{3-[(dimethylamino)methyl]-5-fluorophenoxy}piperidin-1-yl)-3-[4-(7H-pyrrolo[2,3-d]pyrimidin-4-yl)-1H-pyrazol-1-yl]butanenitrile, (S)-3-[1-(6-chloropyridin-2-yl)pyrrolidin-3-yl]-3-[4-(7H-pyrrolo[2,3-d]pyrimidin-4-yl)-1H-pyrazol-1-yl]propanenitrile, (S)-3-(1-[1,3]oxazolo[5,4-b]pyridin-2-ylpyrrolidin-3-yl)-3-[4-(7H-pyrrolo[2,3-d]pyrimidin-4-yl)-1H-pyrazol-1-yl]propanenitrile, (S)-4-[(4-{3-cyano-2-[4-(7H-pyrrolo[2,3-d]pyrimidin-4-yl)-1H-pyrazol-1-yl]propyl}piperazin-1-yl)carbonyl]-3-fluorobenzonitrile, (S)-4-[(4-{3-cyano-2-[3-(7H-pyrrolo[2,3-d]pyrimidin-4-yl)-1H-pyrrol-1-yl]propyl}piperazin-1-yl)carbonyl]-3-fluorobenzonitrile, (S)-4-(4-{3-[(dimethylamino)methyl]-5-fluorophenoxy}piperidin-1-yl)-3-[4-(7H-pyrrolo[2,3-d]pyrimidin-4-yl)-1H-pyrazol-1-yl]butanenitrile; and pharmaceutically acceptable salts of any of the aforementioned.

In some embodiments, the compounds of Table 1 are prepared by the synthetic procedures described in US Patent Publ. No. 2010/0298334, filed May 21, 2010, US Patent Publ. No. 2011/0059951, filed Aug. 31, 2010, US Patent Publ. No. 2011/0224190, filed Mar. 9, 2011, US Patent Publ. No. 2012/0149681, filed Nov. 18, 2011, US Patent Publ. No. 2012/0149682, filed Nov. 18, 2011, US Patent Publ. 2013/0018034, filed Jun. 19, 2012, US Patent Publ. No. 2013/0045963, filed Aug. 17, 2012, and US Patent Publ. No. 2014/0005166, filed May 17, 2013, each of which is incorporated herein by reference in its entirety.

In some embodiments, the selective JAK1 inhibitor is selected from the compounds of US Patent Publ. No. 2010/0298334, filed May 21, 2010, US Patent Publ. No. 2011/0059951, filed Aug. 31, 2010, US Patent Publ. No. 2011/0224190, filed Mar. 9, 2011, US Patent Publ. No. 2012/0149681, filed Nov. 18, 2011, US Patent Publ. No. 2012/0149682, filed Nov. 18, 2011, US Patent Publ. 2013/0018034, filed Jun. 19, 2012, US Patent Publ. No. 2013/0045963, filed Aug. 17, 2012, and US Patent Publ. No. 2014/0005166, filed May 17, 2013, each of which is incorporated herein by reference in its entirety.

In some embodiments, the methods comprise administering from about 15 mg to about 25 mg BID on a free base basis of ruxolitinib, or pharmaceutically acceptable salt thereof, to the patient. In some embodiments, the methods comprise administering from about 10 mg to about 25 mg BID on a free base basis of ruxolitinib, or pharmaceutically acceptable salt thereof, to the patient. In some embodiments, the methods comprise administering from about 15 mg to about 25 mg QD on a free base basis of ruxolitinib, or pharmaceutically acceptable salt thereof, to the patient. In some embodiments, the methods comprise administering from about 10 mg to about 25 mg QD on a free base basis of ruxolitinib, or pharmaceutically acceptable salt thereof, to the patient.

In some embodiments, the JAK inhibitor is a compound disclosed in U.S. Pat. No. 7,598,257, U.S. Pat. No. 7,834,022, US 2009/0233903, US 2010/0298355, US 2011/0207754, US 2010-0298334, US 2011-0059951, US 2011-0224190, US 2012-0149681, US 2012-0149682, US 2013-0018034, US 2013-0045963, U.S. Ser. No. 13/896,802, filed May 17, 2013, U.S. Ser. No. 61/721,308, filed Nov. 1, 2012, or U.S. Ser. No. 61/824,683, filed May 17, 2013, each of which is incorporated herein by reference in its entirety.

The present application further provides a method of predicting a benefit to a patient having a solid tumor of treatment using a JAK inhibitor or an inhibitor of IL-6 signaling, comprising comparing said serum concentration of C-reactive protein (CRP) of the patient to a baseline serum concentration of CRP of a population of patients having the solid tumor, wherein the serum CRP concentration in the patient of equal to or greater than the baseline serum concentration is indicative of a benefit to the patient of the treatment using the JAK inhibitor or an inhibitor of IL-6 signaling.

The present application provides a method of predicting a benefit to a pancreatic cancer patient of treatment using ruxolitinib, or a pharmaceutically acceptable salt thereof, comprising comparing serum concentration of C-reactive protein (CRP) of the patient to a baseline serum concentration of CRP of a population of patients having the solid tumor, wherein the serum CRP concentration in the patient of equal to or greater than the baseline serum concentration is indicative of a benefit to the patient of the treatment using the inhibitor of ruxolitinib, or a pharmaceutically acceptable salt thereof. In some embodiments, the method further comprises measuring the serum concentration of CRP of the patient using a CRP assay prior to said comparing.

In some embodiments, the methods of predicting further comprises measuring the serum concentration of CRP of the patient using a CRP assay prior to said comparing.

In some embodiments, the methods of predicting further comprises prescribing (or administering) a JAK inhibitor or an inhibitor of IL-6 signaling for said patient.

In some embodiments, the benefit is improvement in survival of the patient.

In some embodiments, the benefit is improvement in progression-free survival of the patient.

As used herein, progression-free survival refers to the length of time during and after the treatment of a solid tumor that a patient lives with the disease but it does not get worse.

In some embodiments, the present application provides a method of treating a solid tumor, comprising:

(a) selecting a patient having the solid tumor with a serum concentration of C-reactive protein (CRP) that is equal to or greater than about 10 μg/mL;

(b) administering to the patient a therapeutically effective amount of an inhibitor of ruxolitinib, or a pharmaceutically acceptable salt thereof;

wherein the treating results in increased survival or progression-free survival of the patient.

The present application also provides a method of treating a solid tumor, comprising:

(a) selecting a patient having the solid tumor with a modified Glasgow Prognostic Score of 1 or 2;

(b) administering to the patient a therapeutically effective amount of an inhibitor of ruxolitinib, or a pharmaceutically acceptable salt thereof;

wherein the treating results in increased survival or progression-free survival of the patient.

In some embodiments, the solid tumor referred to in each of the methods is prostate cancer, renal cancer, hepatic cancer, colon cancer, rectal cancer, renal cancer, colorectal cancer, pancreatic cancer, gastric cancer, breast cancer, lung cancer (e.g., metastatic, mesothelioma, or non-small cell lung cancer (NSCLC)), cancers of the head and neck, thyroid cancer, glioblastoma, Kaposi's sarcoma, Castleman's disease, melanoma, oesophageal cancer, gastro-oesophageal cancer, cervical cancer, hepatocellular carcinoma, endometrial cancer, urothelial cancers (e.g., cancer of the bladder ureters and cancer of the renal pelvis, including transitional cell carcinoma (TCC)), or ovarian cancer.

In some embodiments, the solid tumor can further include those characterized by expression of a mutant JAK2 such as those having at least one mutation in the pseudo-kinase domain (e.g., JAK2V617F).

In some embodiments, the solid tumor is pancreatic cancer, prostate cancer, colon cancer, gastric cancer, or lung cancer.

In some embodiments, the solid tumor is pancreatic cancer.

In some embodiments, the solid tumor is pancreatic adenocarcinoma that is recurrent or treatment refractory.

In some embodiments, the solid tumor is metastatic pancreatic adenocarcinoma.

In some embodiments, the solid tumor is advanced pancreatic adenocarcinoma.

In some embodiments, the solid tumor is metastatic pancreatic adenocarcinoma that is recurrent or treatment refractory.

In some embodiments, the solid tumor is advanced pancreatic adenocarcinoma that is recurrent or treatment refractory.

In some embodiments, the solid tumor is prostate cancer.

In some embodiments, the solid tumor is colon cancer.

In some embodiments, the solid tumor is gastric cancer.

In some embodiments, the solid tumor is lung cancer.

In some embodiments, the solid tumor is endometrial cancer.

In some embodiments, the solid tumor is non-small cell lung cancer.

In another aspect, the present application provides a method of increasing survival or progression-free survival in a patient that has diffuse large B-cell lymphoma, wherein the patient has an elevated serum concentration of C-reactive protein (CRP), comprising administering a Janus Kinase (JAK) inhibitor or an inhibitor of IL-6 signaling to the patient, wherein the administering increases survival or progression-free survival of the patient.

The present application also provides a method of increasing survival or progression-free survival in a patient that has diffuse large B-cell lymphoma, wherein the patient has a modified Glasgow Prognostic Score (mGPS) of 1 or 2, comprising administering a JAK inhibitor or an inhibitor of IL-6 signaling to the patient, wherein the administering increases survival or progression-free survival of the patient.

The present application further provides a method of treating diffuse large B-cell lymphoma in a patient in need thereof, wherein the patient modified Glasgow Prognostic Score (mGPS) of 1 or 2, comprising administering a Janus Kinase (JAK) inhibitor or an inhibitor of IL-6 signaling to the patient.

The present application further provides a method of treating diffuse large B-cell lymphoma, comprising:

(a) selecting a patient having the lymphoma with a serum concentration of C-reactive protein (CRP) that is equal to or greater than a median baseline serum concentration of CRP for a population of patients with the solid tumor;

(b) administering to the patient a therapeutically effective amount of a JAK inhibitor or an inhibitor of IL-6 signaling.

The present application also provides a method of treating diffuse large B-cell lymphoma, comprising:

(a) selecting a patient having the lymphoma with a serum concentration of C-reactive protein (CRP) that is equal to or greater than about 10 μg/mL;

(b) administering to the patient a therapeutically effective amount of a JAK inhibitor or an inhibitor of IL-6 signaling.

The present application further provides a method of treating diffuse large B-cell lymphoma, comprising:

(a) selecting a patient having the lymphoma with a modified Glasgow Prognostic Score of 1 or 2;

(b) administering to the patient a therapeutically effective amount of a JAK inhibitor or an inhibitor of IL-6 signaling.

The present application also provides a method of predicting a benefit to a patient having diffuse large B-cell lymphoma of treatment using a JAK inhibitor or an inhibitor of IL-6 signaling, comprising comparing said serum concentration of C-reactive protein (CRP) of the patient to a baseline serum concentration of CRP of a population of patients having the lymphoma, wherein the serum CRP concentration in the patient of equal to or greater than the baseline serum concentration is indicative of a benefit to the patient of the treatment using the JAK inhibitor or an inhibitor of IL-6 signaling.

In some embodiments, the diffuse large B-cell lymphoma is activated B-cell like (ABC) diffuse large B cell lymphoma (ABC-DLBCL). In some embodiments, the diffuse large B-cell lymphoma is germinal center B cell (GCB) diffuse large B cell lymphoma (GCB-DLBCL).

In some embodiments, any of the methods can comprise administering to the patient one or more additional chemotherapeutic agents

In some embodiments, the one or more chemotherapeutic agents are selected from antimetabolite agents, topoisomerase 1 inhibitors, platinum analogs, taxanes, anthracyclines, and EGFR inhibitors, and combinations thereof.

In some embodiments, antimetabolite agents include capecitabine, gemcitabine, and fluorouracil (5-FU).

In some embodiments, taxanes include paclitaxel, Abraxane® (paclitaxel protein-bound particles for injectable suspension), and Taxotere® (docetaxel).

In some embodiments, platinum analogs include oxaliplatin, cisplatin, and carboplatin.

In some embodiments, topoisomerase 1 inhibitors include irinotecan and topotecan.

In some embodiment, anthracyclines include doxorubicin or liposomal formulations of doxorubicin.

In some embodiments, the one or more chemotherapeutic agents are selected from one or more additional chemotherapeutic agents are selected from capecitabine, gemcitabine, Abraxane® (paclitaxel protein-bound particles for injectable suspension), docetaxel, fluorouracil (5-FU), oxaliplatin, cisplatin, carboplatin, irinotecan, topotecan, paclitaxel, leucovorin, doxorubicin, and combinations thereof.

In some embodiments, the chemotherapeutic is FOLFIRINOX (5-FU, leucovorin, irinotecan and oxaliplatin).

In some embodiments, the chemotherapeutic is FOLFOX (folinic acid (leucovorin), 5-FU, and oxaliplatin (Eloxatin).

In some embodiments, the one or more additional chemotherapeutic agents is capecitabine.

In some embodiments, the one or more additional chemotherapeutic agents is capecitabine and oxaloplatin.

In some embodiments, the one or more additional chemotherapeutic agents is fluorouracil (5-FU).

In some embodiments, the one or more additional chemotherapeutic agents is gemcitabine and Abraxane® (paclitaxel protein-bound particles for injectable suspension).

The JAK inhibitors or the inhibitors of IL-6 signaling can include pharmaceutically acceptable salts of the inhibitors. As used herein, “pharmaceutically acceptable salts” refers to derivatives of compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the non-toxic salts of the parent compound formed, for example, from non-toxic inorganic or organic acids. The pharmaceutically acceptable salts can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which includes one or more of the following:

(1) preventing the disease; for example, preventing a disease, condition or disorder in an individual who may be predisposed to the disease, condition or disorder but does not yet experience or display the pathology or symptomatology of the disease;

(2) inhibiting the disease; for example, inhibiting a disease, condition or disorder in an individual who is experiencing or displaying the pathology or symptomatology of the disease, condition or disorder (i.e., arresting further development of the pathology and/or symptomatology), and

(3) ameliorating the disease; for example, ameliorating a disease, condition or disorder in an individual who is experiencing or displaying the pathology or symptomatology of the disease, condition or disorder (i.e., reversing the pathology and/or symptomatology).

### Pharmaceutical Formulations and Dosage Forms

When employed as pharmaceuticals, the JAK inhibitors or inhibitors of IL-6 signaling can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

The methods can also utilize pharmaceutical compositions which contain, as the active ingredient, one or more of JAK inhibitors or inhibitors of IL-6 signaling in combination with one or more pharmaceutically acceptable carriers (excipients). In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions can be formulated in a unit dosage form, each dosage containing from about 5 to about 1000 mg (1 g), more usually about 100 to about 500 mg, about 10 mg, about 15 mg, about 20 mg, or about 25 mg of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient.

The tablets or pills can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the invention can further include one or more additional pharmaceutical agents such as a chemotherapeutic, steroid, anti-inflammatory compound, or immunosuppressant, examples of which are listed hereinabove.

### Combination Therapies

One or more additional pharmaceutical agents such as, for example, chemotherapeutics, anti-inflammatory agents, steroids, immunosuppressants, as well as Bcr-Abl, Flt-3, RAF and FAK kinase inhibitors such as, for example, those described in WO 2006/056399, which is incorporated herein by reference in its entirety, or other agents can be used in combination with the dosage forms described herein for use in the methods described herein. The one or more additional pharmaceutical agents can be administered to a patient simultaneously or sequentially.

In some embodiments, the one or more dosage forms can be used in combination with one or more nonsteroidal anti-inflammatory drugs (NSAIDs). In some embodiments, the NSAIDs are selected from aspirin, diflunisal, salsalate, ibuprofen, naproxen, fenoprofen, ketoprofen, oxaprozin, Indomethicin, tolmetin, sulindac, etodolac, ketodolac, piroxicam, meloxicam, tenoxicam, acetaminophen, celecoxib, and combinations thereof.

Example chemotherapeutics include proteosome inhibitors (e.g., bortezomib), thalidomide, revlimid, and DNA-damaging agents such as melphalan, doxorubicin, cyclophosphamide, vincristine, etoposide, carmustine, and the like.

Example steroids include coriticosteroids such as dexamethasone or prednisone.

Example Bcr-Abl inhibitors include the compounds, and pharmaceutically acceptable salts thereof, of the genera and species disclosed in U.S. Pat. No. 5,521,184, WO 04/005281, and U.S. Ser. No. 60/578,491, all of which are incorporated herein by reference in their entirety.

Example suitable Flt-3 inhibitors include compounds, and their pharmaceutically acceptable salts, as disclosed in WO 03/037347, WO 03/099771, and WO 04/046120, all of which are incorporated herein by reference in their entirety.

Example suitable RAF inhibitors include compounds, and their pharmaceutically acceptable salts, as disclosed in WO 00/09495 and WO 05/028444, both of which are incorporated herein by reference in their entirety.

Example suitable FAK inhibitors include compounds, and their pharmaceutically acceptable salts, as disclosed in WO 04/080980, WO 04/056786, WO 03/024967, WO 01/064655, WO 00/053595, and WO 01/014402, all of which are incorporated herein by reference in their entirety.

In some embodiments, one or more of the dosage forms of the invention can be used in combination with one or more other kinase inhibitors including imatinib, particularly for treating patients resistant to imatinib or other kinase inhibitors.

In some embodiments, one or more dosage forms can be used in combination with a chemotherapeutic in the treatment of a solid tumor and may improve the treatment response as compared to the response to the chemotherapeutic agent alone, without exacerbation of its toxic effects. Examples of additional pharmaceutical agents can include, without limitation, melphalan, melphalan plus prednisone [MP], doxorubicin, dexamethasone, and Velcade (bortezomib). Further additional agents used in the treatment include Bcr-Abl, Flt-3, RAF, mTor, EGFR, PI3K-delta, and FAK kinase inhibitors. Additive or synergistic effects are desirable outcomes of combining a dosage form of the present invention with an additional agent. The agents can be combined with the JAK inhibitors or inhibitors of IL-6 signaling in a single or continuous dosage form, or the agents can be administered simultaneously or sequentially as separate dosage forms.

In some embodiments, a corticosteroid such as dexamethasone is administered to a patient in combination with at the dosage form of the invention where the dexamethasone is administered intermittently as opposed to continuously.

In some further embodiments, combinations of one or more JAK inhibitors or inhibitors of IL-6 signaling with other therapeutic agents can be administered to a patient prior to, during, and/or after a bone marrow transplant or stem cell transplant.

In some embodiments, the additional therapeutic agent is fluocinolone acetonide (Retisert®), or rimexolone (AL-2178, Vexol, Alcon).

In some embodiments, the additional therapeutic agent is cyclosporine (Restasis®).

In some embodiments, the additional therapeutic agent is a corticosteroid. In some embodiments, the corticosteroid is triamcinolone, dexamethasone, fluocinolone, cortisone, prednisolone, or flumetholone.

In some embodiments, the additional therapeutic agent is selected from Dehydrex™ (Holles Labs), Civamide (Opko), sodium hyaluronate (Vismed, Lantibio/TRB Chemedia), cyclosporine (ST-603, Sirion Therapeutics), ARG101(T) (testosterone, Argentis), AGR1012(P) (Argentis), ecabet sodium (Senju-Ista), gefarnate (Santen), 15-(s)-hydroxyeicosatetraenoic acid (15(S)-HETE), cevilemine, doxycycline (ALTY-0501, Alacrity), minocycline, iDestrin™ (NP50301, Nascent Pharmaceuticals), cyclosporine A (Nova22007, Novagali), oxytetracycline (Duramycin, MOLI1901, Lantibio), CF101 (2S,3S,4R,5R)-3,4-dihydroxy-5-[6-[(3-iodophenyl)methylamino]purin-9-yl]-N-methyl-oxolane-2-carbamyl, Can-Fite Biopharma), voclosporin (LX212 or LX214, Lux Biosciences), ARG103 (Agentis), RX-10045 (synthetic resolvin analog, Resolvyx), DYN15 (Dyanmis Therapeutics), rivoglitazone (DE011, Daiichi Sanko), TB4 (RegeneRx), OPH-01 (Ophtalmis Monaco), PCS101 (Pericor Science), REV1-31 (Evolutec), Lacritin (Senju), rebamipide (Otsuka-Novartis), OT-551 (Othera), PAI-2 (University of Pennsylvania and Temple University), pilocarpine, tacrolimus, pimecrolimus (AMS981, Novartis), loteprednol etabonate, rituximab, diquafosol tetrasodium (INS365, Inspire), KLS-0611 (Kissei Pharmaceuticals), dehydroepiandrosterone, anakinra, efalizumab, mycophenolate sodium, etanercept (Embrel®), hydroxychloroquine, NGX267 (TorreyPines Therapeutics), actemra, gemcitabine, oxaliplatin, L-asparaginase, or thalidomide.

In some embodiments, the additional therapeutic agent is an anti-angiogenic agent, cholinergic agonist, TRP-1 receptor modulator, a calcium channel blocker, a mucin secretagogue, MUC1 stimulant, a calcineurin inhibitor, a corticosteroid, a P2Y2 receptor agonist, a muscarinic receptor agonist, an mTOR inhibitor, another JAK inhibitor, Bcr-Abl kinase inhibitor, Flt-3 kinase inhibitor, RAF kinase inhibitor, and FAK kinase inhibitor such as, for example, those described in WO 2006/056399, which is incorporated herein by reference in its entirety. In some embodiments, the additional therapeutic agent is a tetracycline derivative (e.g., minocycline or doxycline). In some embodiments, the additional therapeutic agent binds to FKBP12.

In some embodiments, the additional therapeutic agent is an alkylating agent or DNA cross-linking agent; an anti-metabolite/demethylating agent (e.g., 5-flurouracil, capecitabine or azacitidine); an anti-hormone therapy (e.g., hormone receptor antagonists, SERMs, or aromotase inhibitor); a mitotic inhibitor (e.g. vincristine or paclitaxel); an topoisomerase (I or II) inhibitor (e.g. mitoxantrone and irinotecan); an apoptotic inducers (e.g. ABT-737); a nucleic acid therapy (e.g. antisense or RNAi); nuclear receptor ligands (e.g., agonists and/or antagonists: all-trans retinoic acid or bexarotene); epigenetic targeting agents such as histone deacetylase inhibitors (e.g. vorinostat), hypomethylating agents (e.g. decitabine); regulators of protein stability such as Hsp90 inhibitors, ubiquitin and/or ubiquitin like conjugating or deconjugating molecules; or an EGFR inhibitor (erlotinib).

In some embodiments, the additional therapeutic agent includes an antibiotic, antiviral, antifungal, anesthetic, anti-inflammatory agents including steroidal and non-steroidal anti-inflammatories, and anti-allergic agents. Examples of suitable medicaments include aminoglycosides such as amikacin, gentamycin, tobramycin, streptomycin, netilmycin, and kanamycin; fluoroquinolones such as ciprofloxacin, norfloxacin, ofloxacin, trovafloxacin, lomefloxacin, levofloxacin, and enoxacin; naphthyridine; sulfonamides; polymyxin; chloramphenicol; neomycin; paramomycin; colistimethate; bacitracin; vancomycin; tetracyclines; rifampin and its derivatives (“rifampins”); cycloserine; beta-lactams; cephalosporins; amphotericins; fluconazole; flucytosine; natamycin; miconazole; ketoconazole; corticosteroids; diclofenac; flurbiprofen; ketorolac; suprofen; cromolyn; lodoxamide; levocabastin; naphazoline; antazoline; pheniramine; or azalide antibiotic.

It is further appreciated that certain features of the invention, which are, for clarity, described in the context of separate embodiments, can also be provided in combination in a single embodiment (as if the embodiments of the specification are written as multiply dependent claims).

### EXAMPLES

### Example 1

### Survival Benefit in Pancreatic Patients Having C-Reactive Protein (CRP) Levels Above Median Baseline

The study consisted of an open-label, safety run-in (consisting of 1-2 cohorts) designed to confirm the safety of the capecitabine/ruxolitinib combination this patient population, followed by a randomized, double-blind study with two treatment arms. All subjects received capecitabine therapy in addition to the Study Drug. In the safety run-in, Study Drug was open label ruxolitinib phosphate; for the randomized study, blinded Study Drug was ruxolitinib phosphate or its placebo.

Treatment for subjects consisted of repeating 21 day cycles. Capecitabine was self administered for the first 14 days of each cycle, and the Study Drug was self-administered during the entire cycle. Treatment cycles contineud as long as the regimen was tolerated, and the subject did not meet discontinuation criteria. In the event of disease progression, capecitabine therapy was discontinued; subjects continued to receive Study Drug. Subjects who discontinued treatment with the Study Drug were followed for subsequent treatment regimens and survival.

### Study Design

The parameters of the study conducted by those of skill in the art are described below.

### Safety Run-In:

Cohort 1: 9 subjects will be enrolled to receive capecitabine 2000 mg/m² daily (as 1000 mg/m² BID)+ruxolitinib phosphate at 15 mg BID on a free base basis If 3 or more subjects in Cohort 1 experience a DLT within the first cycle (21 days) of treatment, a second cohort will be enrolled.

Cohort 2: 9 additional subjects will receive capecitabine 2000 mg/m² daily (as 1000 mg/m² BID)+ruxolitinib phosphate at 10 mg BID on a free base basis

In the event that toxicities occurring are clearly associated with capecitabine, a lower dose of capecitabine will be considered rather than, or in addition to, the lower dose of ruxolitinib.

Thus, the dose selected for the randomized portion of the study will be one that is tolerated, without need for dose reduction within 21 days, by at least two-thirds of subjects tested at that dose. If more than 3 DLTs occur in both Cohort 1 and Cohort 2, the randomized portion of the study will not be enrolled.

### Randomized Portion of the Study:

120 subjects randomized 1:1 into 2 treatment arms:

Arm 1: capecitabine+Study Drug (ruxolitinib phosphate)

Arm 2: capecitabine+Study Drug (placebo)

Subjects, Investigators and Sponsor will be blinded to treatment assignment. The starting dose of Study Drug will be one that was selected during the safety run-in.

### Combination Therapy, Dosage and Mode of Administration:

Capecitabine (as open-label, commercial product) will be self-administered as a twice daily (BID) oral treatment for the first 14 days of each cycle. Study Drug (ruxolitinib or placebo) will be self administered as a twice daily (BID) oral treatment during the entire 21 day cycle. Doses defined during the safety run-in will be used in the randomized portion of the study, and individual subjects may have dose reductions of Study Drug or capecitabine during the course of treatment, based upon safety laboratory assessments. Subjects with stable safety parameters may be eligible for dose increase of Study Drug on an individual basis, according to a defined algorithm.
Duration of Participation: Study subject participation is expected to average 4-8 months.
Study Population: Subjects with metastatic pancreatic adenocarcinoma that is recurrent or treatment refractory.

### KEY Inclusion Criteria:

Diagnosis of metastatic pancreatic cancer; subjects must have measurable, or evaluable disease that is histologically confirmed

Karnofsky performance status of ≧60

Subjects must have failed 1st line gemcitabine treatment for metastatic pancreatic cancer:

An alternate chemotherapeutic agent is an acceptable substitute as 1st line therapy in the event that the subject was intolerant to, or ineligible to receive gemcitabine.

≧2 weeks elapsed from the completion of previous chemotherapy, and subjects must have recovered or be at new stable baseline from any related toxicities

### KEY Exclusion Criteria

More than 1 prior chemotherapy regimen (not including adjuvant therapy) for metastatic disease

Evidence of CNS metastases (unless stable for >3 months) or history of uncontrolled seizures

Ongoing radiation therapy or prior radiation therapy administered as a second-line treatment

Other active malignancy except basal or squamous carcinoma of the skin

Inability to swallow food or any condition of the upper GI tract that precludes administration of oral medications

Recent (≦3 months) history of bowel obstruction

Prior severe reaction to fluoropyrimidines, known DPD deficiency, or other known sensitivity to 5-FU

Inadequate renal, hepatic and bone marrow function:

ANC<1500/mm³

Platelets<75,000/mm³

AST/ALT>2.5 X ULN; or >5 X ULN in the presence of liver metastases

Total bilirubin>1.5 X ULN

Creatinine clearance <50 cc/min

### Planned Number of Subjects:

Approximately 9-18 subjects in safety run-in portion of the study, followed by 120 subjects in the randomized portion of the study: 1:1 in each of 2 treatment arms.

### Study Schedule/Procedures:

At Day 1 of each cycle a Study Visit will be conducted to include a physical exam and laboratory tests. Additionally, Laboratory Visits will be conducted weekly during Cycles 1 and 2, and once mid-cycle (approximately Day 10) during all subsequent cycles. Reassessment of tumor size (typically by CT scan) will be conducted every 6 weeks for the duration of study participation. Patient reported outcomes will be collected at some Study Visits. Day 1 of each Study Cycle will correspond with the beginning of the 14-day course of capecitabine. If capecitabine is discontinued, Study Cycles will continue to follow a 21-day repeating schedule. Following discontinuation of all study treatments, assessments will cease, subjects and will be followed for survival and subsequent anti-cancer therapy.
Planned Number of Study Sites: approximately 50
Estimated Study Duration: 20 months
Statistical Methods: Survival data will be analyzed by the Kaplan-Meier method after 95 events. The hazard ratio and its 95% confidence interval will be determined based upon the logrank test and its variance. The sample size of 60 subjects per arm yields a power of 88% to detect a survival difference between Arms 1 and 2 if the true hazard ratio is 0.6. This assumes a one-sided alpha of 0.1, an expected survival of 4 months in the control arm, 8 months of enrollment, and 8 months of follow up after last subject in. There will be a planned interim analysis for futility when half the target number of deaths has accrued.
   Results from Randomized Study

Baseline C-reactive protein (CRP) levels were measured for each patient prior to treatment in Arm 1 and Arm 2. Serum CRP concentrations were measured by RMB (RBM multiplexed Luminex®) commercial assay (Myriad RBM). The subsidiary is Myriad RBM. There are several known commercial clinical assays for determining CRP. The Myriad RBM CRP assay has been shown to correlate with a Luminex CRP assay using commercially available reagents (Millipore) and a clinical Quest Diagnostis CRP assay. The baseline CRP level was calculated on a per patient basis. The patients comprised two groups. Group 1 included all patients who were randomized and took Study Drug. Group 2 are a small subset of patients who passed screening and may or may not have been randomized, but which did not take Study Drug. For Group 1, the CRP level was the last tested CRP level taken before first dose of Study Drug. For Group 2, the last available value of CRP was taken, if available, for example from screening procedures. With baseline CRP levels for all patients, the median was calculated using normal statistical methods known to one skilled in the art. The median baseline CRP for the patient population was 13 μg/mL.

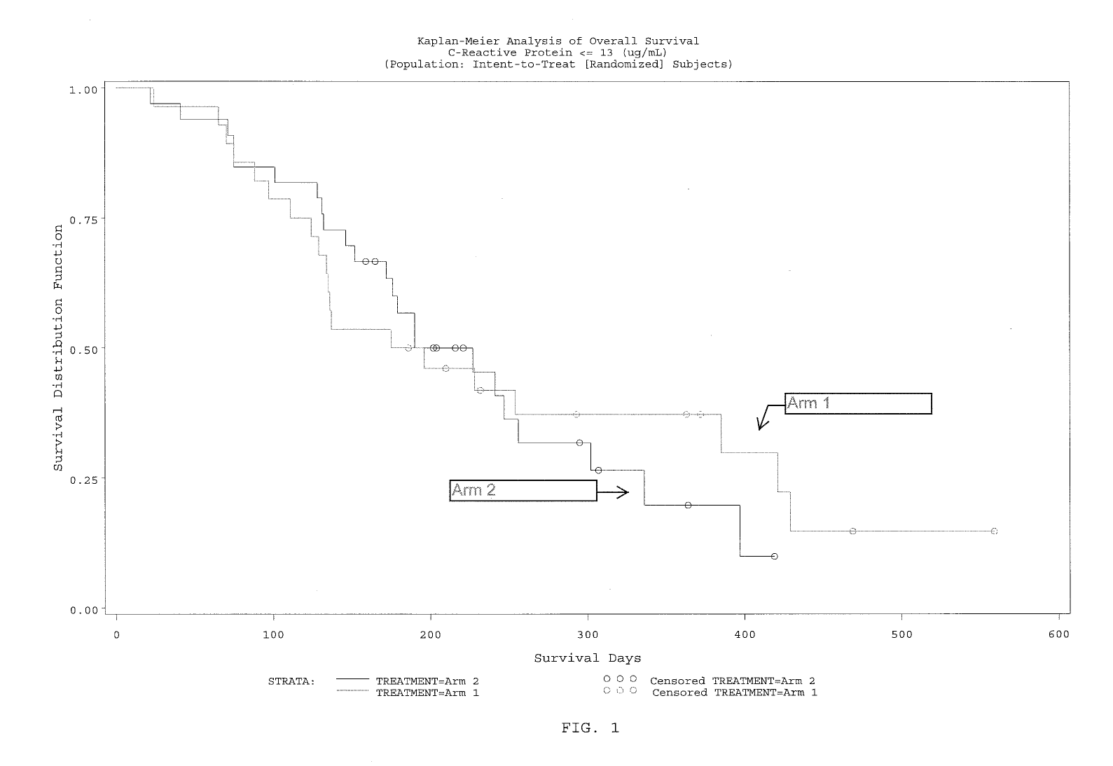

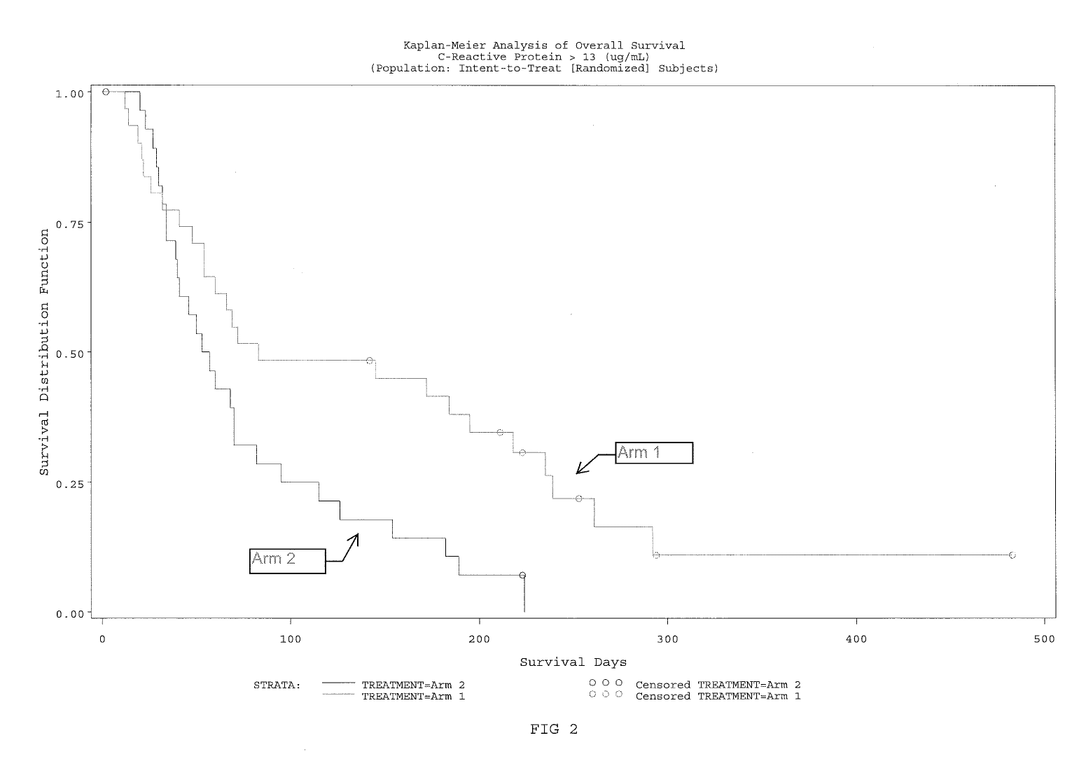

Survival data was analyzed statistically as described using a Kaplan-Meier analysis of overall survival using a score test from Cox Proportional Hazards Model. Table 1 and FIG. 1 shows the results for patients whose baseline CRP was less than or equal to 13 μg/mL, while Table 2 and FIG. 2 shows the results for patients whose baseline CRP was more than 13 μg/mL. Censored subjects were those which were either lost to follow-up or did not have their death recorded prior to the clinical data cut-off.

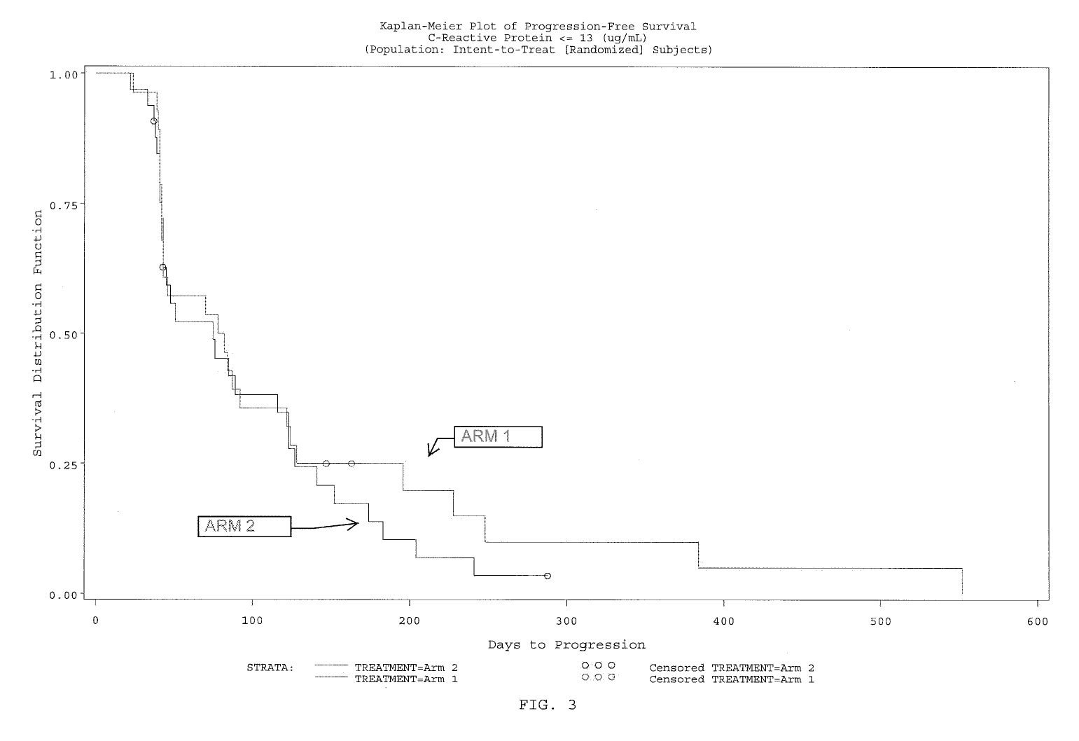

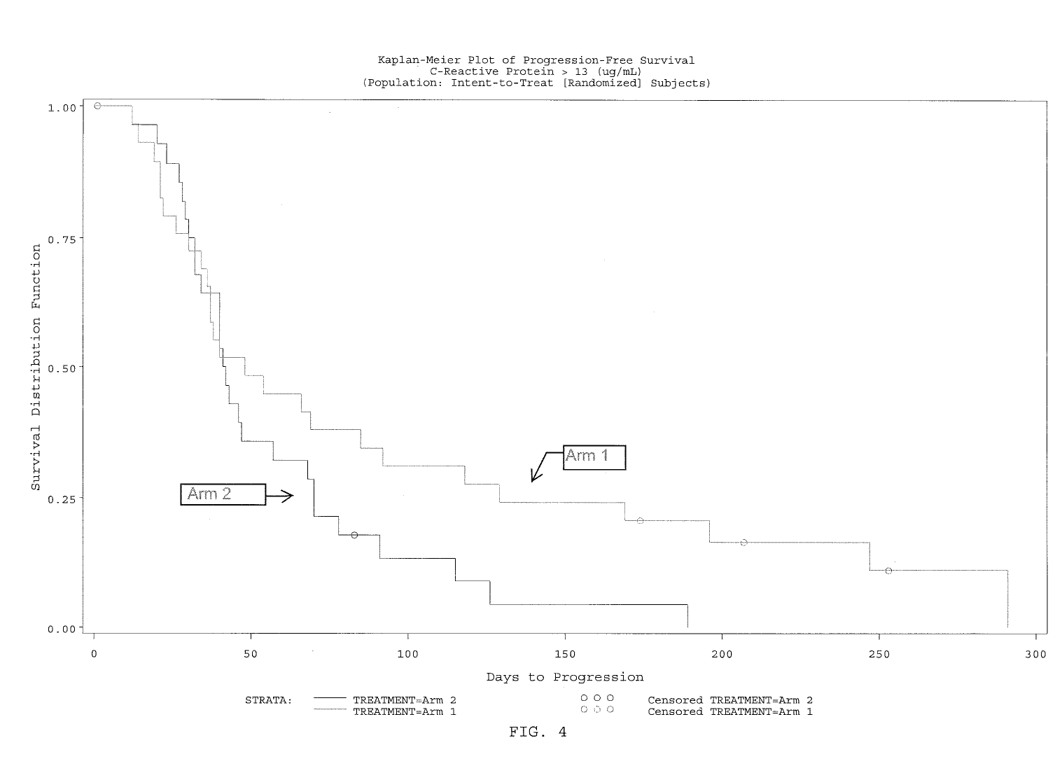

In addition, progression-free survival was analyzed similarly using a Kaplan-Meier analysis of progression-free survival using a score test from Cox Proportional Hazards Model. Table 3 and FIG. 3 shows the results for patients whose baseline CRP was less than or equal to 13 μg/mL, while Table 4 and FIG. 4 shows the results for patients whose baseline CRP was more than 13 μg/mL.

TABLE 1
Kaplan-Meier Analysis of Overall Survival Using Score Test from
Cox Proportional Hazards Model, C-Reactive Protein ≦ 13 (μg/mL)
(Population: Intent-to-Treat [Randomized] Subjects)

| | Variable | | | | |
|---|---|---|---|---|---|
| | Ruxolitinib | | Placebo | | P-value |
| | N = 28 | | N = 33 | | [3] |
| Number (%) of Subjects Died | | | | | |
| Observed | 20 | ( 71.4%) | 23 | ( 69.7%) | 0.3524 |
| Censored | 8 | ( 28.6%) | 10 | ( 30.3%) | |
| Active vs Control Hazard | | | | | 0.887 |
| Ratio (95% CI) [1] | | | | | (0.470, 1.647) |
| Median Time to Event in | 185.5 | (129.0, 385.0) | 208.5 | (152.0, 256.0) | |
| Days (95% CI) [2] | | | | | |
| Month 3 Survival Rate(%) | 82.1 | ( 62.3, 92.1) | 84.8 | ( 67.4, 93.4) | |
| (95% CI) | | | | | |
| Month 6 Survival Rate(%) | 50.0 | ( 30.6, 66.6) | 56.7 | ( 37.9, 71.7) | |
| (95% CI) | | | | | |
| Month 9 Survival Rate(%) | 37.3 | ( 19.4, 55.2) | 31.8 | ( 15.3, 49.8) | |

| | | |
|---|---|---|
| (95% CI) | | |
| Month 12 Survival Rate(%) | 19.9 | ( 6.2, 39.1) |

(95% CI)

[1] The hazard ratio and the 95% CI were estimated using a Cox regression model with Efron's method used for ties.
[2] The median time and the 95% CI were estimated using Brookmeyer and Crowley.
[3] The 1-sided p-value was calculated based on the score test from the Cox Proportional Hazards Model.

TABLE 2
Kaplan-Meier Analysis of Overall Survival Using Score Test from
Cox Proportional Hazards Model, C-Reactive Protein > 13 (μg/mL)
(Population: Intent-to-Treat [Randomized] Subjects)

| | Variable | | | | |
|---|---|---|---|---|---|
| | Ruxolitinib | | Placebo | | P-value |
| | (N = 31) | | (N = 29) | | [3] |
| Number (%) of Subjects Died | | | | | |
| Observed | 25 | ( 80.6%) | 27 | ( 93.1%) | 0.0053 |
| Censored | 6 | ( 19.4%) | 2 | ( 6.9%) | |
| Active vs Control Hazard | | | | | 0.473 |
| Ratio (95% CI) [1] | | | | | (0.260, 0.847) |
| Median Time to Event in | 83.0 | ( 54.0, 218.0) | 55.0 | ( 39.0, 70.0) | |
| Days (95% CI) [2] | | | | | |
| Month 3 Survival Rate(%) | 48.4 | ( 30.2, 64.4) | 28.6 | ( 13.5, 45.6) | |
| (95% CI) | | | | | |
| Month 6 Survival Rate(%) | 41.5 | ( 24.1, 58.0) | 10.7 | ( 2.7, 25.1) | |
| (95% CI) | | | | | |
| Month 9 Survival Rate(%) | 16.5 | ( 5.0, 33.7) | 0.0 | ( , ) | |

| | | |
|---|---|---|
| (95% CI) | | |
| Month 12 Survival Rate(%) | 11.0 | ( 2.2, 27.9) |

(95% CI)

[1] The hazard ratio and the 95% CI were estimated using a Cox regression model with Efron's method used for ties.
[2] The median time and the 95% CI were estimated using Brookmeyer and Crowley.
[3] The 1-sided p-value was calculated based on the score test from the Cox Proportional Hazards Model.

TABLE 3
Kaplan-Meier Analysis of Progression-Free Survival Using Score Test
from Cox Proportional Hazards Model, C-Reactive Protein ≦ 13 (μg/mL)
(Population: Intent-to-Treat [Randomized] Subjects)

| | Variable | | | | |
|---|---|---|---|---|---|
| | Ruxolitinib | | Placebo | | P-value |
| | N = 28 | | N = 33 | | [4] |
| Number (%) of Subjects with | | | | | |
| Disease Progression [1] | | | | | |
| Observed | 26 | ( 92.9%) | 29 | ( 87.9%) | 0.4673 |
| Censored | 2 | ( 7.1%) | 4 | ( 12.1%) | |
| Active vs Control Hazard | | | | | 0.818 |
| Ratio (95% CI) [2] | | | | | (0.471, 1.407) |
| Median Time to Event in | 80.0 | ( 42.0, 122.0) | 75.0 | ( 43.0, 123.0) | |
| Days (95% CI) [3] | | | | | |
| Month 3 Survival Rate(%) | 39.3 | ( 21.7, 56.5) | 38.3 | ( 21.4, 55.0) | |
| (95% CI) | | | | | |
| Month 6 Survival Rate(%) | 25.0 | ( 11.1, 41.8) | 13.9 | ( 4.4, 28.8) | |
| (95% CI) | | | | | |
| Month 9 Survival Rate(%) | 10.0 | ( 1.9, 26.2) | 3.5 | ( 0.3, 15.1) | |

(95% CI)
Month 12 Survival Rate(%)
(95% CI)

[1] Progression free survival was defined as the first occurrence of death or progressive disease by RECIST 1.1
[2] The hazard ratio and the 95% CI were estimated using a Cox regression model with Efron's method used for ties.
[3] The median time and the 95% CI were estimated using Brookmeyer and Crowley.
[4] The 2-sided p-value was calculated based on the score test from the Cox proportional hazards model.

TABLE 4
Kaplan-Meier Analysis of Progression-Free Survival Using Score Test
from Cox Proportional Hazards Model, C-Reactive Protein ≦ 13 (μg/mL)
(Population: Intent-to-Treat [Randomized] Subjects)

| | Variable | | | | |
|---|---|---|---|---|---|
| | Ruxolitinib | | Placebo | | P-value |
| | N = 31 | | N = 29 | | [4] |
| Number (%) of Subjects with | | | | | |
| Disease Progression [1] | | | | | |
| Observed | 26 | ( 83.9%) | 27 | ( 93.1%) | 0.0997 |
| Censored | 5 | ( 16.1%) | 2 | ( 6.9%) | |
| Active vs Control Hazard | | | | | 0.619 |
| Ratio (95% CI) [2] | | | | | (0.345, 1.099) |
| Median Time to Event in | 48.0 | ( 34.0, 92.0) | 41.5 | ( 32.0, 57.0) | |
| Days (95% CI) [3] | | | | | |
| Month 3 Survival Rate(%) | 34.5 | ( 18.2, 51.4) | 13.4 | ( 3.8, 29.0) | |
| (95% CI) | | | | | |
| Month 6 Survival Rate(%) | 20.7 | ( 8.4, 36.7) | 4.5 | ( 0.3, 18.2) | |
| (95% CI) | | | | | |
| Month 9 Survival Rate(%) | 11.0 | ( 2.4, 27.2) | 0.0 | ( , ) | |
| (95% CI) | | | | | |
| Month 12 Survival Rate(%) | 0.0 | ( , ) | | | |
| (95% CI) | | | | | |

[1] Progression free survival was defined as the first occurrence of death or progressive disease by RECIST 1.1
[2] The hazard ratio and the 95% CI were estimated using a Cox regression model with Efron's method used for ties.
[3] The median time and the 95% CI were estimated using Brookmeyer and Crowley.
[4] The 2-sided p-value was calculated based on the score test from the Cox proportional hazards model.

Table 5 shows the result of the Cox regression analysis within the CRP>13 mg/L subgroup. The regression model fits the data well (p=0.022), and accounting for the baseline characteristics in the model, the observed HR in favor of ruxolitinib remains largely preserved (HR 0.50, 95% CI: 0.26-0.96; p=0.037).

TABLE 5
Cox Regression Analysis of Overall Survival Using Baseline
Predictors in Patients With CRP > 13 mg/L

| | | | | | Hazard | Confidence | |
|---|---|---|---|---|---|---|---|
| Predictor | Ruxolitinib | | Placebo | | Ratioⁱ⁾ | Interval | p-valueⁱⁱ⁾ |
| Number (%) subjects who died | | | | | | | |
| Observed | 24 | (80.0) | 27 | (93.1) | | | 0.0218 |
| Censored | 6 | (20.0) | 2 | (6.9) | | | |

| | | | |
|---|---|---|---|
| Treatment (ruxolitinib vs | | | |
| placebo) | 0.501 | (0.259, 0.955) | 0.0369 |
| Age (> 65 years vs ≦ 65 years) | 1.661 | (0.833, 3.439) | 0.1589 |
| LDH (high vs low/normal) | 2.907 | (1.378, 6.329) | 0.0060 |
| ALB (low vs normal/high) | 0.952 | (0.502, 1.791) | 0.8780 |
| Liver metastases (yes vs no) | 0.732 | (0.298, 1.880) | 0.5030 |
| Lung metastases (yes vs no) | 0.671 | (0.314, 1.430) | 0.3014 |
| Karnofsky (60-80 vs 90-100) | 1.582 | (0.834, 3.163) | 0.1745 |
| Prior erlotinib (yes vs no) | 0.181 | (0.049, 0.547) | 0.0052 |
| Prior radiation (yes vs no) | 1.166 | (0.237, 4.660) | 0.8418 |
| Prior Whipple (yes vs no) | 0.830 | (0.180, 4.044) | 0.8168 |
| Sex (male vs female) | 1.550 | (0.726, 3.425) | 0.2661 |

ⁱ⁾The hazard ratio and the 95% CI were estimated using a Cox regression model with Efron's method used for ties.
ⁱⁱ⁾The 2-sided p-value was calculated based on the score test from the Cox proportional hazards model.

Table 6 shows the results of the Cox regression analyses including all of the subgroups above, but with formal interaction testing for the 3 subgroups that were prespecified on the basis of a hypothesis that ruxolitinib would provide a disproportionate benefit. Among these 3 subgroups, only CRP greater than the study population median (>13 mg/L) emerges as a significant factor with a 2-sided Bonferroni-corrected p-value of 0.032.

TABLE 6
Cox Regression Analysis With Formal Interaction Testing

| | Hazard ratio | Unadjusted 2-sided p-value | | Bonferroni Correctedⁱ⁾ | |
|---|---|---|---|---|---|
| Subgroup | (Rux/Pbo) | Cox Regressionⁱⁱ⁾ | Log-Rank | Cox Regression | Log-Rank |
| Low albumin | 0.618 | 0.1786 | 0.1806 | 0.5358 | 0.5418 |
| CRP > 13 mg/L | 0.473 | 0.0106 | 0.0106 | 0.0318 | 0.0318 |
| Karnofsky: 60-80 | 0.799 | 0.4044 | 0.4097 | 1.000 | 1.000 |

ⁱ⁾Based on the Score test, using Efron's method for handling ties.
ⁱⁱ⁾Assumes 3 hypotheses to test.

### Progression Free Survival

In the intent-to-treat analysis including all randomized patients, the HR for progression-free survival (PFS) was 0.75 (CI: 0.52, 1.1, p=0.14).

Assessment of PFS in patients with CRP>13 mg/L, showed a HR of 0.62 (95% CI: 0.35-1.1, p=0.10). The probability of progression-free survival at 3, 6, and 9 months was 35%, 21%, and 11% in the ruxolitinib group and 13%, 5%, and 0% in the placebo group. Assessment of PFS in patients with CRP<13 mg/L, showed a hazard ratio of 0.82 (95% CI: 0.47-1.41, p=0.47). The probability of PFS at 3, 6, and 9 months was 39%, 25%, and 10% in the ruxolitinib group and 38%, 14%, and 4% in the placebo group.

Survival by Modified Glasgow Prognostic Score (mGPS)

The prespecified subgroup analysis used the median CRP for the entire study population (13 mg/L) as a cutoff; however, a post-hoc analysis was conducted using a cutoff of 10 mg/L, consistent with the mGPS) and the generally accepted standard for a clinically meaningful elevation (McMillan et al 2007; FDA Guidance on CRP Assays). For patients with a CRP>10 mg/L (N=70), the HR in favor of ruxolitinib was 0.60 (95% CI: 0.351-1.028, 2-sided p=0.06) and for patients with CRP≦10 mg/L (N=51), the HR was 0.91 (95% CI: 0.46-1.74, p=0.77).

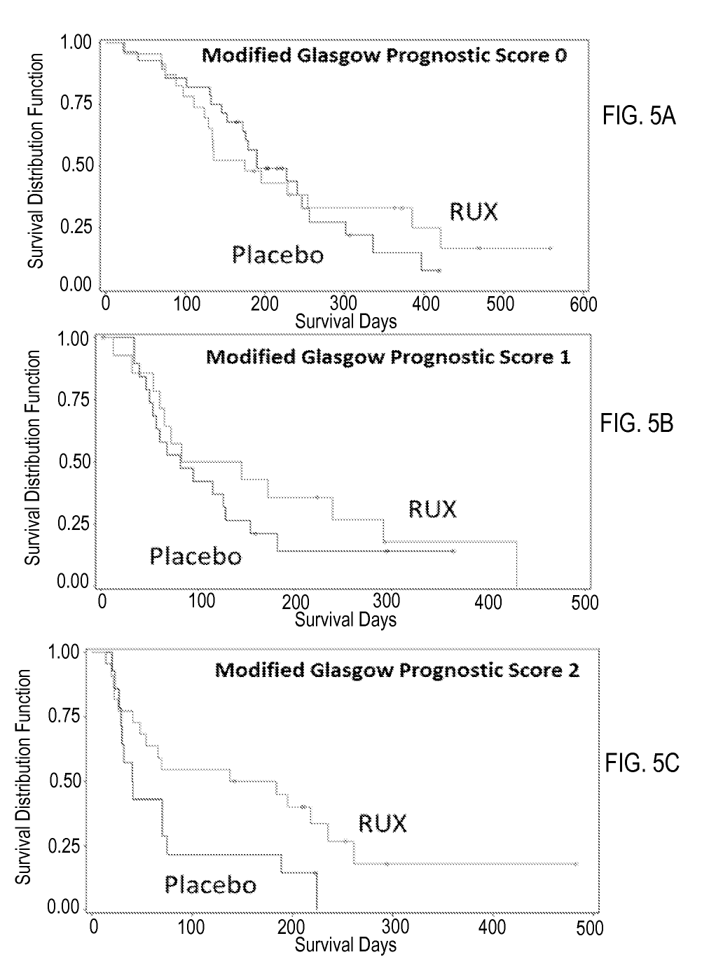

Kaplan-Meier analyses of OS based on the mGPS showed that with increasing mGPS, there was a meaningful separation between the ruxolitinib and placebo groups in OS. For patients with mGPS of 0 (N=51), the HR was 0.91 (95% CI: 0.46-1.74, p=0.77). For patients with mGPS of 1 (N=34), the HR was 0.71 (95% CI: 0.32-1.54, p=0.39). For patients with mGPS of 2 (N=36), the HR was 0.49 (95% CI: 0.23-1.07, p=0.06). Kaplan Meier curves for all 3 groups are presented in FIG. 5.

### Objective Response Rate

A waterfall plot of change in tumor burden as measured by Response Evaluation Criteria in Solid Tumors (RECIST v1.1, sum of single dimensional measures of target lesions) for patients with at least 1 postbaseline tumor assessment showed that patients treated with ruxolitinib were more likely to show tumor shrinkage or stabilization as their best response to therapy. Few patients receiving placebo with CRP>13 mg/L survived long enough to have a postbaseline scan (N=11), and only a few showed response or disease stabilization (36.4%), whereas a larger proportion of patients with CRP>13 mg/L receiving ruxolitinib had postbaseline assessments (N=19), and the majority showed disease stabilization (68.4%).

### Example J1

### ((2R,5S)-5-{2-[(1R)-1-Hydroxyethyl]-1H-imidazo[4,5-d]thieno[3,2-b]pyridin-1-yl}tetrahydro-2H-pyran-2-yl)acetonitrile

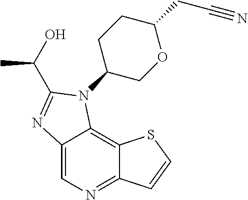 [SMILES: C[C@@H](O)c1nc2cnc3ccsc3c2n1[C@H]1CC[C@H](CC#N)OC1]

### Step 1. tert-Butyl (4S)-2,2-dimethyl-4-vinyl-1,3-oxazolidine-3-carboxylate

To a suspension of methyl triphenylphosphonium bromide (5.63 g, 15.8 mmol) in tetrahydrofuran (140 mL) was added 2.5 M n-butyllithium in hexane (7.35 mL, 18.4 mmol). The deep red solution was stirred at 0° C. for 1 h. Then a solution of tert-butyl (4R)-4-formyl-2,2-dimethyl-1,3-oxazolidine-3-carboxylate (from Aldrich, 3.01 g, 13.1 mmol) in tetrahydrofuran (7.3 mL) was added drop wise at 0° C. The red solution was warmed to room temperature and stirred for 12 h. Hexanes was added to the reaction mixture in 4:1 (v/v) ratio. The suspension was filtered through Celite and the filtrate concentrated. The resultant residue was purified by flash chromatography (eluting with 10% ethyl acetate in hexanes) to give the desired compound as colorless oil (1.92 g, 64%).

### Step 2. tert-Butyl [(1S)-1-(hydroxymethyl)prop-2-en-1-yl]carbamate

To a solution of tert-butyl (4S)-2,2-dimethyl-4-vinyl-1,3-oxazolidine-3-carboxylate (1.90 g, 8.36 mmol) in methanol (83 mL) was added p-toluenesulfonic acid monohydrate (0.80 g, 4.2 mmol) at 0° C. The mixture was slowly warmed to room temperature overnight. The reaction mixture was diluted with saturated NaHCO₃ solution, concentrated, and then diluted with ethyl acetate. The organic layer was washed with sat. NaHCO₃ (2×) and brine, dried over Na₂SO₄, filtered and concentrated to give the desired product as colorless oil (1.187 g, 76%). ¹H NMR (400 MHz, CDCl₃) δ 5.81 (1H, m), 5.25 (2H, m), 4.90 (1H, m), 4.25 (1H, br s), 3.67 (2H, m), 1.45 (9H, s) ppm.

### Step 3. tert-Butyl [(1S)-1-({[1-(hydroxymethyl)prop-2-en-1-yl]oxy}methyl)prop-2-en-1-yl]carbamate

To a flask was charged with tert-butyl [(1S)-1-(hydroxymethyl)prop-2-en-1-yl]carbamate (0.401 g, 2.14 mmol), tris(dibenzylideneacetone)dipalladium(0) (59 mg, 0.064 mmol), N,N′-(1S,2S)-cyclohexane-1,2-diylbis[2-(diphenylphosphino)-1-naphthamide] (150 mg, 0.19 mmol), and 4-dimethylaminopyridine (78 mg, 0.64 mmol). The reaction mixture was purged with N₂ three times, and then methylene chloride (21.3 mL), and 1.0 M triethylborane in THF (130 μL, 0.13 mmol) was added sequentially. After stirring for 10 min, 2-vinyloxirane (0.150 g, 2.14 mmol) was added and the resulting mixture was stirred overnight. The reaction was diluted with dichloromethane and sat. NaHCO₃ solution. The organic layer was separated and dried over Na₂SO₄, filtered and concentrated. The crude residue was purified with flash chromatography (eluting with 0-50% ethyl acetate/hexanes) to give the desired product (0.271 g, 49%). ¹H NMR (300 MHz, CDCl₃) δ 5.85 (1H, m), 5.67 (1H, m), 5.84˜5.17 (4H, m), 4.83 (1H, m), 4.30 (1H, br s), 3.83 (1H, m), 3.69 (1H, dd, J=4.5 and 6.9 Hz), 3.54 (2H, m), 3.36 (1H, dd, J=4.5 and 6.9 Hz), 1.45 (9H, s) ppm.

### Step 4. 2-({(2S)-2-[(tert-Butoxycarbonyl)amino]but-3-en-1-yl}oxy)but-3-en-1-yl acetate

To a mixture of tent-butyl [(1S)-1-({[1-(hydroxymethyl)prop-2-en-1-yl]oxy}methyl)prop-2-en-1-yl]carbamate (268 mg, 1.04 mmol) in methylene chloride (10 mL) was added with triethylamine (435 μL, 3.12 mmol). The mixture was cooled to 0° C., and acetyl chloride (150 μL, 2.1 mmol) was added drop wise. The reaction was stirred at room temperature for 2 h, then quenched with water. The organic layer was concentrated and the resultant residue purified on silica gel (eluting with 20% ethyl acetate/hexanes) to give the desired product (0.26 g, 85%). LCMS calculated for C₁₀H₁₈NO₃ (M−100+H)⁺: m/z=200.1; Found: 200.1.

### Step 5. {(5S)-5-[(tert-Butoxycarbonyl)amino]-5,6-dihydro-2H-pyran-2-yl}methyl acetate

To a 500 mL 2-neck round bottom flask, benzylidene(dichloro)(1,3-dimesitylimidazolidin-2-id-2-yl)(tricyclohexylphosphoranyl)ruthenium (38 mg, 0.044 mmol) was added. After purged with nitrogen for 3 times, dichloromethane (anhydrous, 8 mL) was added followed by 2-({(2S)-2-[(tert-butoxycarbonyl)amino]but-3-en-1-yl}oxy)but-3-en-1-yl acetate (265 mg, 0.885 mmol). The reaction mixture was stirred at room temperature for 15 h. The mixture was concentrated in vacuo. The residue was purified via flash chromatography (eluting with hexanes to 25% EtOAc in hexanes) to give the desired product as a brown oil (0.205 g, 85%). LCMS calculated for C₉H₁₄NO₅ (M+H−Bu+H)⁺: m/z=216.1; Found: 216.1. ¹H NMR (300 MHz, CDCl₃) δ 5.94 (0.17H, m), 5.84 (0.83H, m), 5.69 (1H, m), 4.89 (0.13H, m), 4.70 (0.83H, m), 4.25 (1H, m), 4.05 (4H, m), 3.56 (0.13H, m), 3.38 (0.87H, m), 2.04 (2.49H, s), 2.03 (0.51H, m), 1.38 (9H, s) ppm (The product was a ˜5:1 mixture of trans- and cis-isomers).

### Step 6. [(5S)-5-Amino-5,6-dihydro-2H-pyran-2-yl]methyl acetate

To a solution of {(5S)-5-[(tert-butoxycarbonyl)amino]-5,6-dihydro-2H-pyran-2-yl}methyl acetate (205 mg, 0.756 mmol) in methylene chloride (5.2 mL) was added 4.0 M hydrogen chloride in dioxane (1.5 mL, 6.0 mmol). The reaction solution was stirred at room temperature for 6 h. The solvent was removed under reduced pressure to give the desired product as white solid. LCMS calculated for C₈H₁₄NO₃ (M+H)⁺: m/z=172.1; Found: 172.1.

### Step 7. {(5S)-5-[(6-Nitrothieno[3,2-b]pyridin-7-yl)amino]-5,6-dihydro-2H-pyran-2-yl}methyl acetate

A mixture of 7-chloro-6-nitrothieno[3,2-b]pyridine (156 mg, 0.727 mmol), [(5S)-5-amino-5,6-dihydro-2H-pyran-2-yl]methyl acetate (129 mg, 0.754 mmol) and N,N-diisopropylethylamine (0.26 mL, 1.5 mmol) in isopropyl alcohol (1.7 mL) was heated at 90° C. for 2 h. The reaction mixture was concentrated and purified with flash chromatography to give the desired product (0.21 g 83%). LCMS calculated for C₁₅H₁₆N₃O₅S (M+H)⁺: m/z=350.1; Found: 350.0.

### Step 8. {(5S)-5-[(6-Aminothieno[3,2-b]pyridin-7-yl)amino]tetrahydro-2H-pyran-2-yl}methyl acetate

A mixture of {(5S)-5-[(6-nitrothieno[3,2-b]pyridin-7-yl)amino]-5,6-dihydro-2H-pyran-2-yl}methyl acetate (210 mg, 0.600 mmol) and 10% palladium on carbon (0.21 g) in methanol (4.0 mL) was subjected to balloon pressure of H₂ at room temperature for 2 h. The mixture was filtered, and the filtrate was concentrated and purified with flash chromatography (eluting with 15% methanol in dichloromethane) to give the desired product (145 mg, 75%). LCMS calculated for C₁₅H₂₀N₃O₃S (M+H)⁺: m/z=322.1; Found: 322.0.

### Step 9. (1R)-1-{1-[(3S)-6-(Hydroxymethyl)tetrahydro-2H-pyran-3-yl]-1H-imidazo[4,5-d]thieno[3,2-b]pyridin-2-yl}ethanol

A mixture of (2R)-2-hydroxypropanamide (131 mg, 1.47 mmol) and triethyloxonium tetrafluoroborate (263 mg, 1.38 mmol) in THF (2 mL) was stirred at room temperature for 2 h. The solvent was removed and the residue dissolved in ethanol (0.85 mL) and added to a suspension of {(5S)-5-[(6-aminothieno[3,2-b]pyridin-7-yl)amino]tetrahydro-2H-pyran-2-yl}methyl acetate (145 mg, 0.451 mmol) in ethanol (3.1 mL). The mixture was stirred at 80° C. for 1 h. The reaction was cooled to room temperature and diluted with water (1.0 mL). Lithium hydroxide (32.4 mg, 1.35 mmol) was added, and the mixture was stirred for 2 h. The reaction mixture was diluted with methanol and purified with prep-LCMS (XBridge C18 column, eluting with a gradient of acetonitrile/water containing 0.1% ammonium hydroxide, at flow rate of 60 mL/min) to give the desired product as white solid (95 mg, 63%). LCMS calculated for C₁₆H₂₀N₃O₃S (M+H)⁺: m/z=334.1; Found: 334.0.

### Step 10: ((2R,5S)-5-{2-[(1R)-1-Hydroxyethyl]-1H-imidazo[4,5-d]thieno[3,2-b]pyridin-1-yl}tetrahydro-2H-pyran-2-yl)methyl 4-methylbenzenesulfonate and ((2S,5S)-5-{2-[(1R)-1-hydroxyethyl]-1H-imidazo[4,5-d]thieno[3,2-b]pyridin-1-yl}tetrahydro-2H-pyran-2-yl)methyl 4-methylbenzenesulfonate

To a solution of (1R)-1-{1-[(3S)-6-(hydroxymethyl)tetrahydro-2H-pyran-3-yl]-1H-imidazo[4,5-d]thieno[3,2-b]pyridin-2-yl}ethanol (100 mg, 0.300 mmol) (previous step) in methylene chloride (3.4 mL) and pyridine (0.146 mL, 1.80 mmol) was added p-toluenesulfonyl chloride (57.2 mg, 0.300 mmol) and 4-dimethylaminopyridine (1.8 mg, 0.015 mmol) at 0° C. The reaction mixture was allowed to warm to room temperature overnight. The reaction mixture was concentrated, diluted with methanol, and purified with prep-LCMS (XBridge C18 column, eluting with a gradient of acetonitrile/water containing 0.1% ammonium hydroxide, at flow rate of 60 mL/min) to give two peaks. On analytic HPLC (Waters SunFire C18, 2.1×50 mm, 5 μM; Flow rate 3 mL/min; Injection volume 2 μL; At gradient from 2 to 80% B in 3 minutes (A=water with 0.025% TFA, B=acetonitrile)): First peak (45.3 mg, 31%) retention time 1.81 min, LCMS calculated for C₂₃H₂₆N₃O₅S₂ (M+H)⁺: m/z=488.1; Found: 488.1. Second peak (8.5 mg, 5.8%) retention time 1.88 min, LCMS calculated for C₂₃H₂₆N₃O₅S₂ (M+H)⁺: m/z=488.1; Found: 488.1.

### Step 11. ((2R,5S)-5-{2-[(1R)-1-Hydroxyethyl]-1H-imidazo[4,5-d]thieno[3,2-b]pyridin-1-yl}tetrahydro-2H-pyran-2-yl)acetonitrile

A mixture of ((2R,5S)-5-{2-[(1R)-1-hydroxyethyl]-1H-imidazo[4,5-d]thieno[3,2-b]pyridin-1-yl}tetrahydro-2H-pyran-2-yl)methyl 4-methylbenzenesulfonate (from 1st peak of previous step, 27 mg, 0.055 mmol) and sodium cyanide (4.5 mg, 0.092 mmol) in dimethyl sulfoxide (0.4 mL) was stirred at 50° C. for 4 h. After cooling, the mixture was diluted with methanol and purified with prep-LCMS (XBridge C18 column, eluting with a gradient of acetonitrile/water containing 0.1% ammonium hydroxide, at flow rate of 30 mL/min) to give the desired product (14.5 mg, 76%). LCMS calculated for C₁₇H₁₉N₄O₂S (M+H)⁺: m/z=343.1; Found: 343.0. ¹H NMR (DMSO-d₆, 500 MHz) δ 9.51 (1H, s), 8.45 (1H, d, J=5.5 Hz), 7.97 (1H, d, J=5..5 Hz), 5.31 (1H, m), 5.20 (1H, m), 4.31 (1H, m), 4.23 (1H, m), 4.02 (1H, m), 2.96 (1H, dd, J=17.0 and 4.5 Hz), 2.85 (1H, dd, J=17.0 and 4.5 Hz), 2.66 (1H, m), 2.26 (1H, m), 2.09 (1H, m), 1.73 (1H, m), 1.69 (3H, d, J=6.5 Hz) ppm.

### Example J1a

### (2R,5S)-5-{2-[(1R)-1-Hydroxyethyl]-1H-imidazo[4,5-d]thieno[3,2-b]pyridin-1-yl}tetrahydro-2H-pyran-2-yl)acetonitrile hydrate

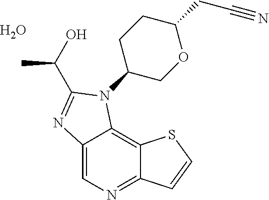

((2R,5S)-5-{2-[(1R)-1-Hydroxyethyl]-1H-imidazo[4,5-d]thieno[3,2-b]pyridin-1-yl}tetrahydro-2H-pyran-2-yl)acetonitrile (52 mg, 0.15 mmol) from Example 25 was crystallized from a mixture of acetonitrile (8 mL) and water (4 mL). The resulting colorless prism crystal collected was suitable for X-ray crystal structure analysis.

Crystal data shows: ˜0.520×0.180×0.100 mm, orthorhombic, P212121, a=6.962(3) Å, b=11.531(4) Å, c=20.799(7) Å, Vol=1669.6(10) Å³, Z=4, T=−100.° C., Formula weight=359.42, Density=1.430 g/cm³, μ(Mo)=0.22 mm⁻¹.

Data collection was done on a Bruker SMART APEX-II CCD system, MoKalpha radiation, standard focus tube, anode power=50 kV×42 mA, crystal to plate distance=5.0 cm, 512×512 pixels/frame, beam center=(256.13,253.14), total frames=1151, oscillation/frame=0.50°, exposure/frame=10.1 sec/frame, SAINT integration, hkl min/max=(−9, 9, −15, 15, −27, 27), data input to shelx=17025, unique data=3975, two-theta range=3.92 to 55.72°, completeness to two-theta 55.72=99.80%, R(int-xl)=0.0681, SADABS correction applied.

Structure was solved using XS(Shelxtl), refined using shelxtl software package, refinement by full-matrix least squares on F², scattering factors from Int. Tab. Vol C Tables 4.2.6.8 and 6.1.1.4, number of data=3975, number of restraints=0, number of parameters=235, data/parameter ratio=16.91, goodness-of-fit on F²=1.04, R indices[I>4sigma(I)] R1=0.0505, wR2=0.1242, R indices(all data) R1=0.0769, wR2=0.1401, max difference peak and hole=0.724 and −0.277 e/Å³, refined flack parameter=−0.12(13), All of the CH hydrogen atoms were refined using a riding model. The OH hydrogens were found from a difference map and fully refined.

Results showed that the asymmetric unit contains one molecule and one water as shown with thermal ellipsoids drawn to the 50% probability level. The stereochemistry at each of three stereocenters (as indicated in the name and structure of the compound above) was confirmed. The flack parameter refined to 0.28(24) indicating the correct enantiomeric setting.

### Example J2

### 4-[3-(Cyanomethyl)-3-(3′,5′-dimethyl-1H,1′H-4,4′-bipyrazol-1-yl)azetidin-1-yl]-2,5-difluoro-N-[(1S)-2,2,2-trifluoro-1-methylethyl]benzamide

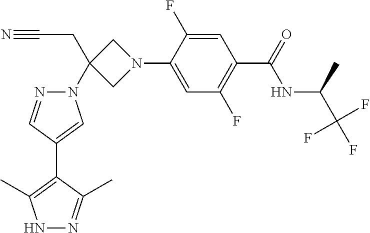 [SMILES: Cc1n[nH]c(C)c1-c1cnn(C2(CC#N)CN(c3cc(F)c(C(=O)N[C@@H](C)C(F)(F)F)cc3F)C2)c1]

### Step 1: 2,4,5-Trifluoro-N-[(1S)-2,2,2-trifluoro-1-methylethyl]benzamide

To a solution of 2,4,5-trifluorobenzoic acid (5.00 g, 28.4 mmol) in acetonitrile (50 mL) was added N,N-dimethylformamide (40 μL) followed by addition of oxalyl chloride (3.60 mL, 42.6 mmol). After 90 min, the volatiles were removed under reduced pressure. The residue was co-evaporated with acetonitrile (50 mL). The residue was then dissolved in methylene chloride (50 mL). This solution was added drop-wise into a cooled (ice bath) mixture of (2S)-1,1,1-trifluoropropan-2-amine hydrochloride (5.52 g, 36.9 mmol) (from Synquest, 98% ee) in toluene (100 mL) and 0.5 M sodium hydroxide aqueous solution (142 mL, 71.0 mmol). After addition, the ice bath was removed, and the reaction was allowed to warm to rt. The reaction was stirred overnight. The organic layer was separated. The aqueous layer was extracted with methylene chloride (50 mL). The combined organic layers were washed with 20% brine (75 mL) and water (2×75 mL), dried over MgSO₄, filtered and concentrated under reduced pressure to afford the desired product (6.49 g, 84%) which was directly used in the next step without further purification. ¹H NMR (300 MHz, DMSO-d₆) δ 9.01 (d, J=7.6 Hz, 1H), 7.92-7.50 (m, 2H), 4.76 (m, 1H), 1.31 (d, J=7.0 Hz, 3H) ppm. LCMS cacld. for C₁₀H₈F₆NO (M+1)⁺: m/z=272.0; Found: 272.0.

### Step 2: 2,5-Difluoro-4-(3-hydroxyazetidin-1-yl)-N-[(1S)-2,2,2-trifluoro-1-methylethyl]benzamide

A mixture of 2,4,5-trifluoro-N-[(1S)-2,2,2-trifluoro-1-methylethyl]benzamide (6.39 g, 23.6 mmol), azetidin-3-ol hydrochloride (3.19 g, 28.3 mmol) and 1,8-diazabicyclo[5.4.0]undec-7-ene (8.81 mL, 58.9 mmol) in acetonitrile (25 mL) was stirred at 80° C. for 2 h. The reaction mixture was diluted with EtOAc (75 mL) and washed with 1N HCl (50 mL), 1N NaHCO₃ (60 mL), 20% brine (50 mL) and water (75 mL). The aqueous layers were extracted with EtOAc (100 mL). The organic layers were combined, dried over MgSO₄, filtered and concentrated under reduced pressure to yield the desired product (7.59 g, 91.8%). ¹H NMR (300 MHz, DMSO-d₆) δ 8.38 (dd, J=8.9, 1.9 Hz, 1H), 7.27 (dd, J=12.8, 6.5 Hz, 1H), 6.38 (dd, J=12.3, 7.5 Hz, 1H), 5.71 (d, J=6.4 Hz, 1H), 4.74 (dp, J=15.3, 7.6 Hz, 1H), 4.62-4.46 (m, 1H), 4.30-4.15 (m, 2H), 3.71 (m, 2H), 1.29 (d, J=7.1 Hz, 3H) ppm. LCMS cacld. for C₁₃H₁₄F₅N₂O₂ (M+1)⁺: m/z=325.1; Found: 325.1.

### Step 3: 2,5-Difluoro-4-(3-oxoazetidin-1-yl)-N-[(1S)-2,2,2-trifluoro-1-methylethyl]benzamide

To a solution of 2,5-difluoro-4-(3-hydroxyazetidin-1-yl)-N-[(1S)-2,2,2-trifluoro-1-methylethyl]benzamide (7.57 g, 23.3 mmol) in methylene chloride (93 mL) was added iodobenzene diacetate (9.40 g, 29.2 mmol) and 2,2,6,6-tetramethyl-1-piperidinyloxy free radical (1.82 g, 11.7 mmol) (TEMPO) at room temperature. The reaction mixture was stirred at room temperature overnight. The mixture was diluted with EtOAc (100 mL), washed with 0.5N NaHCO₃ (2×80 mL), 20% brine (100 mL) and water (100 mL). The aqueous layers were extracted with ethyl acetate (75 mL). The organic extracts were combined, dried over MgSO₄, filtered and concentrated under reduced pressure. The residue was purified by flash chromatography on a silica gel column eluting with 0% to 5% ethyl acetate in methylene chloride to afford the crude product which was recrystallized from MTBE (50 mL) and heptane (100 mL) to give the desired product (5.44 g, 72%) as colorless solid. ¹H NMR (300 MHz, DMSO-d₆) δ 8.52 (d, J=8.0 Hz, 1H), 7.36 (dd, J=12.5, 6.5 Hz, 1H), 6.63 (dd, J=12.1, 7.6 Hz, 1H), 4.90 (d, J=2.1 Hz, 4H), 4.86-4.68 (m, 1H), 1.31 (d, J=7.1 Hz, 3H) ppm. LCMS cacld. for C₁₃H₁₂F₅N₂O₂ (M+1)⁺: m/z=323.1; Found: 323.0.

### Step 4: 4-[3-(Cyanomethylene)azetidin-1-yl]-2,5-difluoro-N-[(1S)-2,2,2-trifluoro-1-methylethyl]benzamide

Diethyl cyanomethylphosphonate (1.95 mL, 11.8 mmol) was added drop-wise to a cooled (ice bath) solution of 1.0 M potassium tert-butoxide in THF (11.8 mL, 11.8 mmol) which was diluted with tetrahydrofuran (12 mL). The bath was removed and the reaction was warmed to room temperature, and stirred for 90 min. The reaction solution was cooled with an ice bath again. The above prepared solution was then added over 12 min to a cooled (ice-bath) solution of 2,5-difluoro-4-(3-oxoazetidin-1-yl)-N-[(1S)-2,2,2-trifluoro-1-methylethyl]benzamide (4.00 g, 12.4 mmol) in tetrahydrofuran (50 mL). The reaction mixture was stirred for 30 min. The ice bath was removed, and the reaction was stirred at room temperature overnight, then quenched by the addition of 20% brine (75 mL) and ethyl acetate (75 mL). The organic layer was separated. The aqueous layer was extracted with ethyl acetate (50 mL). The combined organic layers were dried over MgSO₄, filtered and concentrated under reduced pressure. The residue was purified by flash chromatography on a silica gel column with ethyl acetate in hexanes (0% to 30%) to yield the desired product (2.6 g). ¹H NMR (400 MHz, DMSO-d₆) δ 8.59-8.37 (m, 1H), 7.33 (dd, J=12.5, 6.4 Hz, 1H), 6.59 (dd, J=12.0, 7.4 Hz, 1H), 5.88 (m, 1H), 4.94-4.75 (m, 4H), 4.76 (m, 1H), 1.31 (d, J=7.1 Hz, 3H) ppm. LCMS cacld. for C₁₅H₁₃F₅N₃O (M+1)⁺: m/z=346.1; Found: 346.1.

### Step 5: 4-{3-(Cyanomethyl)-3-[4-(4,4,5,5-tetramethyl-1,3,2-dioxaborolan-2-yl)-1H-pyrazol-1-yl]azetidin-1-yl}-2,5-difluoro-N-[(1S)-2,2,2-trifluoro-1-methylethyl]benzamide

A mixture of 4-(4,4,5,5-tetramethyl-1,3,2-dioxaborolan-2-yl)-1H-pyrazole (1.00 g, 5.15 mmol), 4-[3-(cyanomethylene)azetidin-1-yl]-2,5-difluoro-N-[(1S)-2,2,2-trifluoro-1-methylethyl]benzamide (1.78 g, 5.15 mmol) and 1,8-diazabicyclo[5.4.0]undec-7-ene (0.31 mL, 2.1 mmol) in acetonitrile (20.2 mL) was heated at 50° C. overnight. After cooling, the solvent was removed under reduced pressure. The residue was used in the next step without further purification. LCMS cacld. for C₂₄H₂₈BF₅N₅O₃ (M+1)⁺: m/z=540.2; Found: 540.1.

### Step 6: 4-[3-(Cyanomethyl)-3-(3′,5′-dimethyl-1H,1′H-4,4′-bipyrazol-1-yl)azetidin-1-yl]-2,5-difluoro-N-[(1S)-2,2,2-trifluoro-1-methylethyl]benzamide

A mixture of 4-{3-(cyanomethyl)-3-[4-(4,4,5,5-tetramethyl-1,3,2-dioxaborolan-2-yl)-1H-pyrazol-1-yl]azetidin-1-yl}-2,5-difluoro-N-[(1S)-2,2,2-trifluoro-1-methylethyl]benzamide (329 mg, 0.610 mmol), 4-bromo-3,5-dimethyl-1H-pyrazole (206 mg, 1.18 mmol), tetrakis(triphenylphosphine)palladium(0) (110 mg, 0.098 mmol) and sodium carbonate (320 mg, 3.0 mmol) in 1,4-dioxane (10 mL)/water (5 mL) was purged with nitrogen and stirred at 110° C. for 1 h. The reaction mixture was diluted with EtOAc, washed with water and brine, concentrated. The residue was purified first with silica gel (eluting with 0-100% EtOAc/hexanes followed by 10% methanol/dichloromethane), and then by prep-LCMS (XBridge C18 column, eluting with a gradient of acetonitrile/water containing 0.1% ammonium hydroxide, at flow rate of 60 mL/min) to give the desired product (30 mg, 9.7%). ¹H NMR (500 MHz, DMSO-d₆) δ 12.17 (1H, s), 8.45 (1H, d, J=8.0 Hz), 8.10 (1H, s), 7.70 (1H, s), 7.34 (1H, m), 6.61 (1H, s), 4.77 (1H, m), 4.62 (2H, d, J=9.0 Hz), 4.39 (1H, d, J=9.0 Hz), 3.64 (2H, s), 2.22 (6H, s), 1.31 (6H, d, J=7.0 Hz) ppm. LCMS calculated for C₂₃H₂₃F₅N₇O (M+H)⁺: m/z=508.2; Found: 508.0.

### Example A

### In vitro JAK Kinase Assay

The compound of Formula I herein was tested for inhibitory activity of JAK targets according to the following in vitro assay described in Park et al., Analytical Biochemistry 1999, 269, 94-104. The catalytic domains of human JAK1 (a.a. 837-1142) and JAK2 (a.a. 828-1132) with an N-terminal His tag were expressed using baculovirus in insect cells and purified. The catalytic activity of JAK1 and JAK2 was assayed by measuring the phosphorylation of a biotinylated peptide. The phosphorylated peptide was detected by homogenous time resolved fluorescence (HTRF). IC₅₀s of compounds were measured for each kinase in the 40 microL reactions that contain the enzyme, ATP and 500 nM peptide in 50 mM Tris (pH 7.8) buffer with 100 mM NaCl, 5 mM DTT, and 0.1 mg/mL (0.01%) BSA. For the 1 mM IC₅₀ measurements, ATP concentration in the reactions was 1 mM. Reactions were carried out at room temperature for 1 hr and then stopped with 20 μL 45 mM EDTA, 300 nM SA-APC, 6 nM Eu-Py20 in assay buffer (Perkin Elmer, Boston, Mass.). Binding to the Europium labeled antibody took place for 40 minutes and HTRF signal was measured on a Fusion plate reader (Perkin Elmer, Boston, Mass.).

### Example B

### Cellular Assays

Cancer cell lines dependent on cytokines and hence JAK/STAT signal transduction, for growth, can be plated at 6000 cells per well (96 well plate format) in RPMI 1640, 10% FBS, and 1 nG/mL of appropriate cytokine. Compounds can be added to the cells in DMSO/media (final concentration 0.2% DMSO) and incubated for 72 hours at 37° C., 5% CO₂. The effect of compound on cell viability is assessed using the CellTiter-Glo Luminescent Cell Viability Assay (Promega) followed by TopCount (Perkin Elmer, Boston, Mass.) quantitation. Potential off-target effects of compounds are measured in parallel using a non-JAK driven cell line with the same assay readout. All experiments are typically performed in duplicate.

The above cell lines can also be used to examine the effects of compounds on phosphorylation of JAK kinases or potential downstream substrates such as STAT proteins, Akt, Shp2, or Erk. These experiments can be performed following an overnight cytokine starvation, followed by a brief preincubation with compound (2 hours or less) and cytokine stimulation of approximately 1 hour or less. Proteins are then extracted from cells and analyzed by techniques familiar to those schooled in the art including Western blotting or ELISAs using antibodies that can differentiate between phosphorylated and total protein. These experiments can utilize normal or cancer cells to investigate the activity of compounds on tumor cell survival biology or on mediators of inflammatory disease. For example, with regards to the latter, cytokines such as IL-6, IL-12, IL-23, or IFN can be used to stimulate JAK activation resulting in phosphorylation of STAT protein(s) and potentially in transcriptional profiles (assessed by array or qPCR technology) or production and/or secretion of proteins, such as IL-17. The ability of compounds to inhibit these cytokine mediated effects can be measured using techniques common to those schooled in the art.

Compounds herein can also be tested in cellular models designed to evaluate their potency and activity against mutant JAKs, for example, the JAK2V617F mutation found in myeloid proliferative disorders. These experiments often utilize cytokine dependent cells of hematological lineage (e.g. BaF/3) into which the wild-type or mutant JAK kinases are ectopically expressed (James, C., et al. Nature 434:1144-1148; Staerk, J., et al. JBC 280:41893-41899). Endpoints include the effects of compounds on cell survival, proliferation, and phosphorylated JAK, STAT, Akt, or Erk proteins.

Certain compounds herein can be evaluated for their activity inhibiting T-cell proliferation. Such as assay can be considered a second cytokine (i.e. JAK) driven proliferation assay and also a simplistic assay of immune suppression or inhibition of immune activation. The following is a brief outline of how such experiments can be performed. Peripheral blood mononuclear cells (PBMCs) are prepared from human whole blood samples using Ficoll Hypaque separation method and T-cells (fraction 2000) can be obtained from PBMCs by elutriation. Freshly isolated human T-cells can be maintained in culture medium (RPMI 1640 supplemented with10% fetal bovine serum, 100 U/ml penicillin, 100 μg/ml streptomycin) at a density of 2×10⁶ cells/ml at 37° C. for up to 2 days. For IL-2 stimulated cell proliferation analysis, T-cells are first treated with Phytohemagglutinin (PHA) at a final concentration of 10 μg/mL for 72 h. After washing once with PBS, 6000 cells/well are plated in 96-well plates and treated with compounds at different concentrations in the culture medium in the presence of 100 U/mL human IL-2 (ProSpec-Tany TechnoGene; Rehovot, Israel). The plates are incubated at 37° C. for 72h and the proliferation index is assessed using CellTiter-Glo Luminescent reagents following the manufactory suggested protocol (Promega; Madison, Wis.).

### Example C

### In vivo Anti-Tumor Efficacy

Compounds herein can be evaluated in human tumor xenograft models in immune compromised mice. For example, a tumorigenic variant of the INA-6 plasmacytoma cell line can be used to inoculate SCID mice subcutaneously (Burger, R., et al. Hematol J. 2:42-53, 2001). Tumor bearing animals can then be randomized into drug or vehicle treatment groups and different doses of compounds can be administered by any number of the usual routes including oral, i.p., or continuous infusion using implantable pumps. Tumor growth is followed over time using calipers. Further, tumor samples can be harvested at any time after the initiation of treatment for analysis as described above (Example B) to evaluate compound effects on JAK activity and downstream signaling pathways. In addition, selectivity of the compound(s) can be assessed using xenograft tumor models that are driven by other know kinases (e.g. Bcr-Abl) such as the K562 tumor model.

### Example D

### Murine Skin Contact Delayed Hypersensitivity Response Test

Compounds herein can also be tested for their efficacies (of inhibiting JAK targets) in the T-cell driven murine delayed hypersensitivity test model. The murine skin contact delayed-type hypersensitivity (DTH) response is considered to be a valid model of clinical contact dermatitis, and other T-lymphocyte mediated immune disorders of the skin, such as psoriasis (Immunol Today. 1998 January; 19(1):37-44). Murine DTH shares multiple characteristics with psoriasis, including the immune infiltrate, the accompanying increase in inflammatory cytokines, and keratinocyte hyperproliferation. Furthermore, many classes of agents that are efficacious in treating psoriasis in the clinic are also effective inhibitors of the DTH response in mice (Agents Actions. 1993 January; 38(1-2):116-21).

On Day 0 and 1, Balb/c mice are sensitized with a topical application, to their shaved abdomen with the antigen 2,4,dinitro-fluorobenzene (DNFB). On day 5, ears are measured for thickness using an engineer's micrometer. This measurement is recorded and used as a baseline. Both of the animals' ears are then challenged by a topical application of DNFB in a total of 20 μL (10 μL on the internal pinna and 10 μL on the external pinna) at a concentration of 0.2%. Twenty-four to seventy-two hours after the challenge, ears are measured again. Treatment with the test compounds is given throughout the sensitization and challenge phases (day −1 to day 7) or prior to and throughout the challenge phase (usually afternoon of day 4 to day 7). Treatment of the test compounds (in different concentration) is administered either systemically or topically (topical application of the treatment to the ears). Efficacies of the test compounds are indicated by a reduction in ear swelling comparing to the situation without the treatment. Compounds causing a reduction of 20% or more were considered efficacious. In some experiments, the mice are challenged but not sensitized (negative control).

The inhibitive effect (inhibiting activation of the JAK-STAT pathways) of the test compounds can be confirmed by immunohistochemical analysis. Activation of the JAK-STAT pathway(s) results in the formation and translocation of functional transcription factors. Further, the influx of immune cells and the increased proliferation of keratinocytes should also provide unique expression profile changes in the ear that can be investigated and quantified. Formalin fixed and paraffin embedded ear sections (harvested after the challenge phase in the DTH model) are subjected to immunohistochemical analysis using an antibody that specifically interacts with phosphorylated STAT3 (clone 58E12, Cell Signaling Technologies). The mouse ears are treated with test compounds, vehicle, or dexamethasone (a clinically efficacious treatment for psoriasis), or without any treatment, in the DTH model for comparisons. Test compounds and the dexamethasone can produce similar transcriptional changes both qualitatively and quantitatively, and both the test compounds and dexamethasone can reduce the number of infiltrating cells. Both systemically and topical administration of the test compounds can produce inhibitive effects, i.e., reduction in the number of infiltrating cells and inhibition of the transcriptional changes.

### Example E

### In vivo Anti-Inflammatory Activity

Compounds herein can be evaluated in rodent or non-rodent models designed to replicate a single or complex inflammation response. For instance, rodent models of arthritis can be used to evaluate the therapeutic potential of compounds dosed preventatively or therapeutically. These models include but are not limited to mouse or rat collagen-induced arthritis, rat adjuvant-induced arthritis, and collagen antibody-induced arthritis. Autoimmune diseases including, but not limited to, multiple sclerosis, type I-diabetes mellitus, uveoretinitis, thyroditis, myasthenia gravis, immunoglobulin nephropathies, myocarditis, airway sensitization (asthma), lupus, or colitis may also be used to evaluate the therapeutic potential of compounds herein. These models are well established in the research community and are familiar to those schooled in the art (Current Protocols in Immunology, Vol 3., Coligan, J. E. et al, Wiley Press.; Methods in Molecular Biology: Vol. 225, Inflammation Protocols., Winyard, P. G. and Willoughby, D. A., Humana Press, 2003.).

Each of the journal or patent references supra is incorporated herein by reference in its entirety.

1. A method of increasing survival or progression-free survival in a patient that has a solid tumor, wherein the patient has elevated serum concentrations of C-reactive protein (CRP), comprising administering a JAK inhibitor or an inhibitor of IL-6 signaling to the patient, wherein the administering increases survival or progression-free survival of the patient.

2. The method of claim 1, wherein the method further comprises selecting a patient with an elevated serum concentration of C-reactive protein prior to said administering.

3. The method of claim 1, wherein the method further comprises
selecting a patient having the solid tumor with a serum concentration of C-reactive protein (CRP) that is equal to or greater than a median baseline serum concentration of CRP for a population of patients with the solid tumor.

4. The method of claim 1, wherein the method further comprises
selecting a patient having the solid tumor with a serum concentration of C-reactive protein (CRP) that is equal to or greater than about 10 μg/mL.

5-6. (canceled)

7. The method of claim 1, wherein the serum concentration of CRP is equal to or greater than about 13 μg/mL.

8. A method of treating a solid tumor or diffuse large B-cell lymphoma in a patient in need thereof, wherein the patient modified Glasgow Prognostic Score (mGPS) of 1 or 2, comprising administering a Janus Kinase (JAK) inhibitor or an inhibitor of IL-6 signaling to the patient.

9. The method of claim 1, wherein the method further comprises
selecting a patient having the solid tumor with a modified Glasgow Prognostic Score of 1 or 2.

10. The method of claim 8, wherein the method is a method of treating a solid tumor.

11. The method of claim 8, wherein the method is a method of treating diffuse large B-cell lymphoma.

12. The method of claim 10, wherein the solid tumor is prostate cancer, renal cancer, hepatic cancer, colon cancer, rectal cancer, renal cancer, colorectal cancer, pancreatic cancer, gastric cancer, breast cancer, lung cancer, cancers of the head and neck, thyroid cancer, glioblastoma, Kaposi's sarcoma, melanoma, oesophageal cancer, gastro-oesophageal cancer, cervical cancer, hepatocellular carcinoma, endometrial cancer, urothelial cancer, or ovarian cancer.

13. The method of claim 10, wherein the solid tumor is prostate cancer, pancreatic cancer, gastric cancer, colon cancer, or lung cancer.

14. The method of claim 10, wherein the solid tumor is pancreatic cancer.

15. The method of claim 10, wherein the solid tumor is endometrial cancer.

16. The method of claim 10, wherein the solid tumor is the solid tumor is non-small cell lung cancer.

17. The method of claim 10, wherein the method comprises administering a JAK inhibitor to the patient.

18. The method of claim 10, wherein the method comprises administering an inhibitor of IL-6 signaling to the patient.

19. The method of claim 10, wherein the JAK inhibitor is ruxolitinib, or a pharmaceutically acceptable salt thereof.

20. The method of claim 10, wherein the method comprises administering from about 15 mg to about 25 mg BID on a free base basis of ruxolitinib, or pharmaceutically acceptable salt thereof, to the patient.

21. The method of claim 10, wherein the method further comprises administering to the patient one or more additional chemotherapeutic agents.

22. The method of claim 21, wherein the one or more chemotherapeutic agents are selected from antimetabolite agents, topoisomerase 1 inhibitors, platinum analogs, taxanes, and anthracyclines, EGFR inhibitors, and combinations thereof.

23. The method of claim 21, wherein the one or more additional chemotherapeutic agents are selected from capecitabine, gemcitabine, Abraxane® (paclitaxel protein-bound particles for injectable suspension), docetaxel, fluorouracil (5-FU), oxaliplatin, cisplatin, carboplatin, irinotecan, topotecan, paclitaxel, leucovorin, doxorubicin, and combinations thereof.

24. The method of claim 21, wherein the one or more additional chemotherapeutic agents is capecitabine.

25. A method of predicting a benefit to a patient having a solid tumor of treatment using a JAK inhibitor or an inhibitor of IL-6 signaling, comprising comparing said serum concentration of C-reactive protein (CRP) of the patient to a baseline serum concentration of CRP of a population of patients having the solid tumor, wherein the serum CRP concentration in the patient of equal to or greater than the baseline serum concentration is indicative of a benefit to the patient of the treatment using the JAK inhibitor or an inhibitor of IL-6 signaling.

26. The method of claim 25, wherein the method further comprises measuring the serum concentration of CRP of the patient using a CRP assay prior to said comparing.

27. The method of claim 25, wherein the method further comprises prescribing a JAK inhibitor or an inhibitor of IL-6 signaling for said patient.

28. The method of claim 25, wherein said benefit is improvement in overall survival of the patient.

29. The method of claim 25, wherein said benefit is improvement in progression-free survival of the patient.

30-39. (canceled)

40. A method of predicting a benefit to a pancreatic cancer patient of treatment using ruxolitinib, or a pharmaceutically acceptable salt thereof, comprising comparing serum concentration of C-reactive protein (CRP) of the patient to a baseline serum concentration of CRP of a population of patients having the solid tumor, wherein the serum CRP concentration in the patient of equal to or greater than the baseline serum concentration is indicative of a benefit to the patient of the treatment using the inhibitor of ruxolitinib, or a pharmaceutically acceptable salt thereof.

41-47. (canceled)

48. The method of claim 10, wherein the JAK inhibitor is a selective JAK1 inhibitor.

49. The method of claim 48, wherein said selective JAK1 inhibitor is selected from:
3-[1-(6-chloropyridin-2-yl)pyrrolidin-3-yl]-3-[4-(7H-pyrrolo[2,3-d]pyrimidin-4-yl)-1H-pyrazol-1-yl]propanenitrile;
3-(1-[1,3]oxazolo[5,4-b]pyridin-2-ylpyrrolidin-3-yl)-3-[4-(7H-pyrrolo[2,3-d]pyrimidin-4-yl)-1H-pyrazol-1-yl]propanenitrile;
4-[(4-{3-cyano-2-[4-(7H-pyrrolo[2,3-d]pyrimidin-4-yl)-1H-pyrazol-1-yl]propyl}piperazin-1-yl)carbonyl]-3-fluorobenzonitrile;
4-[(4-{3-cyano-2-[3-(7H-pyrrolo[2,3-d]pyrimidin-4-yl)-1H-pyrrol-1-yl]propyl}piperazin-1-yl)carbonyl]-3-fluorobenzonitrile;
{1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile;
4-{3-(cyanomethyl)-3-[4-(7H-pyrrolo[2,3-d]pyrimidin-4-yl)-1H-pyrazol-1-yl]azetidin-1-yl}-N-[4-fluoro-2-(trifluoromethyl)phenyl]piperidine-1-carboxamide;
[3-[4-(7H-pyrrolo[2,3-d]pyrimidin-4-yl)-1H-pyrazol-1-yl]-1-(1-{[2-(trifluoromethyl)pyrimidin-4-yl]carbonyl}piperidin-4-yl)azetidin-3-yl]acetonitrile;
[trans-1-[4-(7H-pyrrolo[2,3-d]pyrimidin-4-yl)-1H-pyrazol-1-yl]-3-(4-{[2-(trifluoromethyl)pyrimidin-4-yl]carbonyl}piperazin-1-yl)cyclobutyl]acetonitrile;
{trans-3-(4-{[4-[(3-hydroxyazetidin-1-yl)methyl]-6-(trifluoromethyl)pyridin-2-yl]oxy}piperidin-1-yl)-1-[4-(7H-pyrrolo[2,3-d]pyrimidin-4-yl)-1H-pyrazol-1-yl]cyclobutyl}acetonitrile;
{trans-3-(4-{[4-{[(2S)-2-(hydroxymethyl)pyrrolidin-1-yl]methyl}-6-(trifluoromethyl)pyridin-2-yl]oxy}piperidin-1-yl)-1-[4-(7H-pyrrolo[2,3-d]pyrimidin-4-yl)-1H-pyrazol-1-yl]cyclobutyl}acetonitrile;
{trans-3-(4-{[4-{[(2R)-2-(hydroxymethyl)pyrrolidin-1-yl]methyl}-6-(trifluoromethyl)pyridin-2-yl]oxy}piperidin-1-yl)-1-[4-(7H-pyrrolo[2,3-d]pyrimidin-4-yl)-1H-pyrazol-1-yl]cyclobutyl}acetonitrile;
4-(4-{3-[(dimethylamino)methyl]-5-fluorophenoxy}piperidin-1-yl)-3-[4-(7H-pyrrolo[2,3-d]pyrimidin-4-yl)-1H-pyrazol-1-yl]butanenitrile;
5-{3-(cyanomethyl)-3-[4-(7H-pyrrolo[2,3-d]pyrimidin-4-yl)-1H-pyrazol-1-yl]azetidin-1-yl}-N-isopropylpyrazine-2-carboxamide;
4-{3-(cyanomethyl)-3-[4-(7H-pyrrolo[2,3-d]pyrimidin-4-yl)-1H-pyrazol-1-yl]azetidin-1-yl}-2,5-difluoro-N-[(1S)-2,2,2-trifluoro-1-methylethyl]benzamide;
5-{3-(cyanomethyl)-3-[4-(1H-pyrrolo[2,3-b]pyridin-4-yl)-1H-pyrazol-1-yl]azetidin-1-yl}-N-isopropylpyrazine-2-carboxamide;
{1-(cis-4-{[6-(2-hydroxyethyl)-2-(trifluoromethyl)pyrimidin-4-yl]oxy}cyclohexyl)-3-[4-(7H-pyrrolo[2,3-d]pyrimidin-4-yl)-1H-pyrazol-1-yl]azetidin-3-yl}acetonitrile;
{1-(cis-4-{[4-[(ethylamino)methyl]-6-(trifluoromethyl)pyridin-2-yl]oxy}cyclohexyl)-3-[4-(7H-pyrrolo[2,3-d]pyrimidin-4-yl)-1H-pyrazol-1-yl]azetidin-3-yl}acetonitrile;
{1-(cis-4-{[4-(1-hydroxy-1-methylethyl)-6-(trifluoromethyl)pyridin-2-yl]oxy}cyclohexyl)-3-[4-(7H-pyrrolo[2,3-d]pyrimidin-4-yl)-1H-pyrazol-1-yl]azetidin-3-yl}acetonitrile;
{1-(cis-4-{[4-{[(3R)-3-hydroxypyrrolidin-1-yl]methyl}-6-(trifluoromethyl)pyridin-2-yl]oxy}cyclohexyl)-3-[4-(7H-pyrrolo[2,3-d]pyrimidin-4-yl)-1H-pyrazol-1-yl]azetidin-3-yl}acetonitrile;
{1-(cis-4-{[4-{[(3S)-3-hydroxypyrrolidin-1-yl]methyl}-6-(trifluoromethyl)pyridin-2-yl]oxy}cyclohexyl)-3-[4-(7H-pyrrolo[2,3-d]pyrimidin-4-yl)-1 H-pyrazol-1-yl]azetidin-3-yl}acetonitrile;
{trans-3-(4-{[4-({[(1S)-2-hydroxy-1-methylethyl]amino}methyl)-6-(trifluoromethyl)pyridin-2-yl]oxy}piperidin-1-yl)-1-[4-(7H-pyrrolo[2,3-d]pyrimidin-4-yl)-1H-pyrazol-1-yl]cyclobutyl}acetonitrile;
{trans-3-(4-{[4-({[(2R)-2-hydroxypropyl]amino}methyl)-6-(trifluoromethyl)pyridin-2-yl]oxy}piperidin-1-yl)-1-[4-(7H-pyrrolo[2,3-d]pyrimidin-4-yl)-1H-pyrazol-1-yl]cyclobutyl}acetonitrile;
{trans-3-(4-{[4-({[(2S)-2-hydroxypropyl]amino}methyl)-6-(trifluoromethyl)pyridin-2-yl]oxy}piperidin-1-yl)-1-[4-(7H-pyrrolo[2,3-d]pyrimidin-4-yl)-1H-pyrazol-1-yl]cyclobutyl}acetonitrile;
{trans-3-(4-{[4-(2-hydroxyethyl)-6-(trifluoromethyl)pyridin-2-yl]oxy}piperidin-1-yl)-1-[4-(7H-pyrrolo[2,3-d]pyrimidin-4-yl)-1H-pyrazol-1-yl]cyclobutyl}acetonitrile;
((2R,5S)-5-{2-[(1R)-1-hydroxyethyl]-1H-imidazo[4,5-d]thieno[3,2-b]pyridin-1-yl}tetrahydro-2H-pyran-2-yl)acetonitrile; and
4-[3-(cyanomethyl)-3-(3′,5′-dimethyl-1H,1′H-4,4′-bipyrazol-1-yl)azetidin-1-yl]-2,5-difluoro-N-[(1S)-2,2,2-trifluoro-1-methylethyl]benzamide;
or a pharmaceutically acceptable salt of any of the aforementioned.

50. The method of claim 48, wherein the selective JAK1 inhibitor is ((2R,5S)-5-{2-[(1R)-1-hydroxyethyl]-1H-imidazo[4,5-d]thieno[3,2-b]pyridin-1-yl}tetrahydro-2H-pyran-2-yl)acetonitrile, or a pharmaceutically acceptable salt thereof.

51. The method of claim 48, wherein the selective JAK1 inhibitor is 4-[3-(cyanomethyl)-3-(3′,5′-dimethyl-1H, 1′H-4,4′-bipyrazol-1-yl)azetidin-1-yl]-2,5-difluoro-N-[(1S)-2,2,2-trifluoro-1-methylethyl]benzamide, or a pharmaceutically acceptable salt thereof.

52. The method of claim 48, wherein the selective JAK1 inhibitor is {1-{1-[3-fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile, or a pharmaceutically acceptable salt thereof.

53. The method of claim 10, further comprising administering one or more nonsteroidal anti-inflammatory drugs (NSAIDs) to said patient.

54. The method of claim 53, wherein the NSAIDs are selected from aspirin, diflunisal, salsalate, ibuprofen, naproxen, fenoprofen, ketoprofen, oxaprozin, Indomethicin, tolmetin, sulindac, etodolac, ketodolac, piroxicam, meloxicam, tenoxicam, acetaminophen, celecoxib and combinations thereof.

55. The method of claim 10, wherein the administering increases survival in a patient.

56. The method of claim 10, wherein the administering increases progression-free survival of the patient.
